(19) United States
(12) Patent Application Publication
(10) Pub. No.: US 2024/0008428 A1
(43) Pub. Date: Jan. 11, 2024

(54) APPARATUS AND METHOD FOR CULTURING SPHAGNUM

(71) Applicant: MICROPROPAGATION SERVICES (E.M.) LIMITED, Loughborough Leicestershiree (GB)

(72) Inventor: Neal Wright, Loughborough Leicestershire (GB)

(73) Assignee: MICROPROPAGATION SERVICES (E.M.) LIMITED, Loughborough Leicestershire (GB)

(21) Appl. No.: 18/038,428

(22) PCT Filed: Nov. 24, 2021
(86) PCT No.: PCT/GB21/53047

§ 371 Date: May 23, 2023

(30) Foreign Application Priority Data
Nov. 24, 2020 (GB) 2018485.9

(51) Int. Cl. A01G 22/30; A01G 9/18; A01H 4/00; A01H 11/00

(52) U.S. Cl. CPC A01G 22/30; A01G 9/18; A01H 4/002; A01H 11/00

(57) ABSTRACT

An apparatus (100) for use in culturing Sphagnum (106) is provided. The apparatus (100) comprises a culture vessel (102) for Sphagnum (106) and a culture medium (104) arranged in the culture vessel (102). The culture vessel (102) comprises an enclosed airspace (108) above the culture medium (104), and Sphagnum (106) arranged in the culture medium (104). The apparatus (100) further comprises means (110, 112) for supplying carbon dioxide into the enclosed airspace (108).

The present disclosure relates to Sphagnum, in particular to an apparatus for use in culturing Sphagnum. The present disclosure also relates to methods of culturing Sphagnum.

Sphagnum is a genus of moss. It is a lower plant, or a non-vascular plant, and is an example of a bryophyte. It is often referred to as peat moss and typically grows in the wild in peatlands or wetlands. Examples of suitable habitats for Sphagnum include bogs, such as raised bogs and blanket bogs, moors, mires, and fens. Sphagnum has a particularly high capacity for maintaining water in its hyaline cells. As such, in its natural environment, Sphagnum typically grows in wet conditions such as in peatlands.

Peatlands around the world are formed when lower layers of Sphagnum decay to form peat, while the upper layer continues to grow on the surface. As a result of this, carbon is stored within the peat while the actively-growing upper Sphagnum continues sequestering carbon dioxide from the atmosphere. Peatlands cover approximately 3% of the land on the Earth's surface, but store over 500 Gigatonnes of carbon—more than all other vegetation types combined. However, due to adverse impacts on the peatlands (e.g. industrial pollution, drainage—particularly for agriculture, and peat harvesting) the actively-growing upper Sphagnum has been eroded (or is now absent) in many peatlands, thereby exposing the peat to the atmosphere. This absence of surface Sphagnum enables carbon to evaporate from the peatland. This is a pressing environmental issue, and damaged peatlands now contribute around 6% of global anthropogenic carbon dioxide emissions. As a result, there is a pressing need for effective peatland restoration and methods of effectively growing Sphagnum for restoration purposes. Conventional methods of peatland restoration typically involve translocating Sphagnum from other sites including peatlands, which is clearly not sustainable.

Recently, efforts have been made to cultivate Sphagnum sustainably, such as through in vitro tissue culture techniques. Carbon is essential to growth of Sphagnum through photosynthesis. Many techniques for culturing Sphagnum are based on those of other plants, and conventionally provide sugar as a carbon source, generally in the form of sucrose added to a nutrient medium. This is considered advantageous because the sugar can be supplied in a closed system, where the Sphagnum wants to be sealed from the environment to avoid contamination during tissue culture. However, providing sucrose results in ideal growth conditions for contaminants, which then tend to dominate and out-compete Sphagnum. Sphagnum is known as a particularly difficult plant to sterilise in order to remove contaminants (especially because of its high water content in hyaline cells, from which contaminants are difficult to remove), and the resultant culture of Sphagnum generally contains many contaminants which dominate the Sphagnum when cultured in the presence of sugar. This effect is particularly significant for Sphagnum because Sphagnum is adapted to grow in harsh conditions, such as in peatlands with little or no nutrient and sugar availability. Thus, it has adapted to out-compete other organisms at low nutrient and sugar levels. However, where an abundance of sugar is supplied during cultivation of Sphagnum, it has been found to promote unwanted growth of contaminants, which can then out-compete the Sphagnum. There is therefore desired an alternative method of culturing Sphagnum.

The present disclosure seeks to address one or more of the above problems.

The invention is defined by the appended independent claims, while optional features are set out in the appended dependent claims.

According to a first aspect of the present disclosure, there is provided an apparatus for use in culturing Sphagnum, comprising: a culture vessel for Sphagnum; a culture medium arranged in the culture vessel, wherein the culture vessel comprises an enclosed airspace above the culture medium; Sphagnum arranged in the culture medium; and means for supplying carbon dioxide into the enclosed airspace.

In contrast to the typical supply of carbon for photosynthesis in the form of sugar (e.g. sucrose) in a growth medium, the inventors have found that supplying carbon in the form of gaseous carbon dioxide can be beneficial for Sphagnum. Supplying carbon in the form of carbon dioxide enables the necessary levels of carbon to be supplied for growth of Sphagnum, while avoiding a source for contaminant growth such as bacteria or fungi (as is the result of using sugar such as in the form of sucrose). Therefore, supplying carbon dioxide can reduce the contamination of cultures, and thereby lead to an overall improvement in growth, as well as a reduction in cost as fewer contaminated cultures are wasted. Contamination can also stunt or prevent the growth of Sphagnum, so avoiding contamination can improve growth rates.

As used herein, “culturing Sphagnum” is preferably to be understood to mean maintaining Sphagnum in conditions suitable for growth. In a preferred embodiment, culturing comprises growing the Sphagnum. Specifically, culturing the Sphagnum can preferably refer to growing the Sphagnum under in vitro conditions. In other words, preferably the apparatus is for use in in vitro culturing Sphagnum. Accordingly, a “culture vessel for Sphagnum” is a container which is suitable for culturing Sphagnum.

The culture medium is suitable for culturing Sphagnum. The culture medium may otherwise be referred to as a growth medium. In some examples, the culture medium comprises nutrients, and in this case may be referred to as a nutrient medium.

The enclosed airspace is preferably to be understood as a region of the interior volume of the culture vessel, arranged above the culture medium. In other words, the enclosed airspace may be the remainder of the culture vessel that is not filled with culture medium. Thus, the culture medium partially fills the culture vessel. The culture vessel encloses the airspace such that the airspace is not open to the environment. In some examples, the culture vessel may have a lid, or is otherwise closed such that the airspace is isolated from the external environment outside of the culture vessel. This prevents ingress of contamination into the culture vessel. In some examples, the culture vessel is sealed except for the supply of carbon dioxide.

The means for supplying carbon dioxide is preferably configured to supply carbon dioxide into the enclosed airspace of the culture vessel. Thus, the airspace is enclosed preferably except for the means for supplying carbon dioxide. Because the carbon dioxide is supplied to the enclosed airspace, the Sphagnum is not exposed to the outside environment, and contamination is avoided, for example compared to having an open container of Sphagnum.

The apparatus of the present disclosure therefore provides a culture vessel for culturing Sphagnum, and means for supplying carbon dioxide into the culture vessel. This provides an alternative carbon source to sugar, which can help avoid contamination, or at least significantly reduce such levels of contamination.

By supplying the carbon dioxide to the enclosed airspace, the carbon dioxide can be directed to raise the concentration of carbon dioxide in the air above the culture medium. The carbon dioxide can then be absorbed by the Sphagnum. This is advantageous compared to supplying sugar as it can reduce levels of contamination.

Disclosed herein is an apparatus for use in culturing Sphagnum, comprising: a culture vessel for Sphagnum configured to hold a culture medium and Sphagnum in the culture medium, wherein the culture vessel comprises an enclosed airspace above the culture medium; and means for supplying carbon dioxide into the enclosed airspace.

Disclosed herein is an apparatus for use in culturing Sphagnum, comprising: a culture vessel for Sphagnum; a culture medium arranged in the culture vessel, wherein the culture vessel comprises an enclosed airspace above the culture medium; Sphagnum arranged in the culture medium; and a conduit for supplying carbon dioxide into the enclosed airspace.

The apparatus may further comprise means for supplying light to the culture vessel to provide light to the Sphagnum for photosynthesis. This can further improve growth rates.

In some embodiments, the Sphagnum may be in the form of strands of whole plants of Sphagnum. In other examples, the Sphagnum may be in the form of fragments of Sphagnum.

Preferably, the Sphagnum is in vitro Sphagnum. This means that the Sphagnum has been grown under in vitro conditions. More preferably, the Sphagnum is from a micropropagated source. In particular, the Sphagnum is preferably initiated using micropropagation techniques, and cultured in vitro. For example, preferably the Sphagnum is initiated from a vegetative fragment, most preferably from a capitulum. In other examples, the Sphagnum may be initiated from spores. Preferably, the Sphagnum may be surface cleaned or sterilised before entering the culture vessel.

In some embodiments, the culture medium comprises a solid culture medium. For example, the solid culture medium may contain agar to solidify the medium. In this case, the Sphagnum can be arranged on the solid culture medium. In this case, in other words, the term “Sphagnum arranged in the culture medium” should preferably be interpreted to mean that the Sphagnum is arranged within or preferably on top of the solid culture medium. In other words, the solid culture medium may support the Sphagnum on its upper surface. Using a solid culture medium can be beneficial to isolate contaminants. This may be used at an early stage in the culturing process. For example, Sphagnum may be initiated on a solid culture medium because then contaminants can be visibly identified and removed more easily than in liquid culture media. Once the Sphagnum becomes large enough and is visibly clean, it can be transferred into a liquid culture medium which can allow for faster growth due to an increased supply of nutrients to the Sphagnum. Sphagnum does not have roots, and instead nutrients can be supplied over its surface area. Preferably, the Sphagnum is grown in vitro in a solid culture medium, optionally for at least one month, and is then transferred to an apparatus with a liquid culture medium. With the solid medium, the carbon dioxide supplied to the enclosed airspace can then be taken up by the Sphagnum resting on the solid culture medium. By raising the concentration of carbon dioxide in the enclosed airspace, the uptake of carbon dioxide can be increased while avoiding contamination.

Preferably, the culture medium comprises a liquid culture medium. In this case, the Sphagnum can be arranged within the liquid culture medium. In order words, the Sphagnum may be dispersed within the liquid culture medium. In cases where the culture medium is a liquid culture medium, the apparatus has particular advantages and as such it is preferable compared to using a solid culture medium. The carbon dioxide supplied to the enclosed airspace above the liquid culture medium can diffuse into the liquid culture medium at the boundary between the airspace and the liquid culture medium. The higher the concentration of carbon dioxide in the airspace, the greater the diffusion gradient, and thus the greater the uptake of carbon dioxide by the liquid culture medium. In this way, the carbon dioxide can be indirectly supplied into the liquid culture medium via the airspace. The Sphagnum within the liquid culture medium can then absorb the carbon dioxide from the liquid culture medium. As the Sphagnum is surrounded by the liquid culture medium, the amount of carbon dioxide supplied can be relatively high.

In this way, the apparatus can be configured to supply carbon dioxide indirectly to the liquid culture medium. Supplying indirectly should be differentiated from supplying directly. In other words, the means for supplying carbon dioxide supplies carbon dioxide into the enclosed airspace rather than directly into the liquid culture medium. Carbon dioxide can then be absorbed from the airspace into the liquid culture medium indirectly. In contrast, supplying carbon dioxide directly into the liquid culture medium can have several drawbacks. For example, heavy foam formation can result due to the bubbling effect of supplying carbon dioxide directly to the liquid culture medium (e.g. aerating the liquid culture medium). Instead, indirectly supplying carbon dioxide to the airspace avoids foam formation. This also permits the carbon dioxide to diffuse into the liquid culture medium over a large surface area (defined by the boundary between the enclosed airspace and the liquid culture medium i.e. typically the cross-sectional area of the culture vessel). The apparatus of the present disclosure is therefore advantageous compared to aeration methods.

Said another way, the means for supplying carbon dioxide can be configured to supply carbon dioxide directly to the enclosed airspace. In order words, the means for supplying carbon dioxide can be configured to release carbon dioxide into the enclosed airspace. This is to be differentiated from releasing carbon dioxide into the liquid culture medium. For example, the carbon dioxide may be released into the enclosed airspace via an inlet in the culture vessel. The airspace may be otherwise enclosed except for the inlet attached to the supply of carbon dioxide. This is to be differentiated from supplying carbon dioxide directly to the liquid culture medium.

Supplying carbon dioxide to an enclosed airspace is preferable to raising the carbon dioxide levels in an entire room. Providing elevated levels of carbon dioxide in a room can be dangerous and must be monitored closely with complex and expensive equipment, while safety precautions such as alarms must be used. Large amounts of carbon dioxide are also required which is expensive and inefficient. In contrast, raising the concentration of carbon dioxide in the enclosed airspace of the culture vessels provides a safer, cheaper, more efficient way of supplying carbon dioxide to the culture vessels. Also, because the culture vessel are not open, the risk of contamination is much lower.

Optionally, the means for supplying carbon dioxide is configured to supply a gas comprising at least 1% carbon dioxide by volume. In other words, at least 1% of the volume of the gas supplied is carbon dioxide. In other words, the gas supplied comprises at least 10,000 ppm carbon dioxide. This is much higher than atmospheric levels of around 0.04%. It is desirable to provide a high concentration of carbon dioxide in the enclosed airspace. In some embodiments, the means for supplying carbon dioxide is configured to supply a gas containing carbon dioxide. For example, the gas may be air. However, air has low levels of carbon dioxide which limits the ability to supply desired levels of carbon dioxide to the Sphagnum. Instead, the gas may have elevated levels of carbon dioxide. This means that the levels of carbon dioxide have been deliberately increased. For example, the gas (such as air) may be treated or otherwise mixed with carbon dioxide to provide a gas with a greater carbon dioxide content (such as greater than in air). By providing at least 1% carbon dioxide, a higher concentration of carbon dioxide can be supplied which improves rates of photosynthesis and thus can lead to better growth. Especially where a permeable barrier is used, it is beneficial to supply a higher percentage of carbon dioxide than air so that sufficient carbon dioxide is provided for improved growth.

Preferably, the means for supplying carbon dioxide is configured to supply a gas comprising at least 2% carbon dioxide by volume. More preferably, the means for supplying carbon dioxide is configured to supply a gas containing at least 5% carbon dioxide by volume, more preferably at least 10%, even more preferably at least 50%, still more preferably at least 75%. Yet still more preferably, the means for supplying carbon dioxide is configured to supply a gas comprising at least 90% carbon dioxide by volume. In a most preferred embodiment, the means for supplying carbon dioxide is configured to supply at least 99% carbon dioxide by volume. At such levels, the carbon dioxide supplied may be referred to as pure carbon dioxide or substantially pure carbon dioxide. For example, the means for supplying carbon dioxide can be in the form of a canister of carbon dioxide. This provides a much greater supply of carbon dioxide than, for example, supplying air. The volume and flow rate of the gas supplied may be varied depending on the concentration of carbon dioxide in order to supply the required amount of carbon dioxide.

This pure form can also contain fewer contaminants than air, which improves the sterility. It has been found that optimum growth can be achieved with at least 90% carbon dioxide. Additionally, this has been found to be beneficial as this provides a high level of carbon dioxide to diffuse over the permeable barrier and provide a high concentration in the enclosed airspace.

In some examples, the carbon dioxide is supplied at a higher pressure than atmospheric pressure. For example, the carbon dioxide may be supplied through a tube, wherein the pressure of the carbon dioxide (or the gas containing the carbon dioxide) is higher than surrounding air at atmospheric pressure. This provides a mechanism for actively supplying carbon dioxide into the culture vessel.

Optionally, the means for supplying carbon dioxide comprises a source of carbon dioxide. In one example, the source of carbon dioxide is a container holding carbon dioxide. For instance, it may be a canister of compressed carbon dioxide, in liquid or gaseous form. In other examples, the carbon dioxide may be a mixture of gases containing carbon dioxide, such as air with an elevated level of carbon dioxide. Preferably, the source of carbon dioxide comprises a pressurised container of carbon dioxide. In other words, the apparatus may comprise a source of carbon dioxide for supplying carbon dioxide into the enclosed airspace.

Optionally, the means for supplying carbon dioxide comprises a conduit arrangement configured to convey carbon dioxide into the enclosed airspace. For example, the conduit arrangement may provide a path for supplying carbon dioxide from the source of carbon dioxide to the enclosed airspace. In some examples, the conduit arrangement is arranged between the source of carbon dioxide and the enclosed airspace. In some examples, the apparatus may comprise a conduit arrangement configured to convey carbon dioxide into the enclosed airspace. The conduit arrangement may comprise one or more pipes to carry the carbon dioxide from the source of carbon dioxide to the enclosed airspace.

Optionally, the means for supplying carbon dioxide comprises an inlet pipe connected to a source of carbon dioxide. For example, the conduit arrangement may comprise an inlet pipe. The inlet pipe is preferably a conduit for conveying a fluid, specifically for conveying carbon dioxide. Thus, carbon dioxide is able to flow along and within the inlet pipe. The inlet pipe may have a hollow interior for carrying the carbon dioxide, and an external, preferably tubular, surface containing the interior therein. The inlet pipe may have a circular cross-section such that the external surface is generally cylindrical, at least when straight. The inlet pipe may be flexible such that it can be bent along its length, and thus the external surface may not be cylindrical in use. In other examples, the inlet pipe may be other shapes, and may have other cross-sections, including square or rectangular. A first end of the inlet pipe may be connected to the source of carbon dioxide.

In some examples, the inlet pipe is configured to convey the carbon dioxide from the source of carbon dioxide into the culture vessel. In other words, the enclosed airspace can be in fluid communication with the source of carbon dioxide, such as via the inlet pipe. The inlet pipe can have one end connected to the source of carbon dioxide, while the other is arranged to supply the carbon dioxide to the enclosed airspace. In other examples, the inlet pipe may supply carbon dioxide to another pipe or conduit which in turn delivers the carbon dioxide into the enclosed airspace (for example, through a permeable barrier). In some examples, the source of carbon dioxide provides the carbon dioxide which is carried by the inlet pipe to the culture vessel.

In some examples, the apparatus further comprises means for controlling the flow of carbon dioxide from the source of carbon dioxide into the culture vessel. For example, the means for controlling the flow may comprise a valve. The means for controlling the flow may be arranged within the conduit arrangement, within the inlet pipe, connected to the source of carbon dioxide, and/or connected to the culture vessel at the end of the inlet pipe or other conduit of the conduit arrangement. For example, this may allow the supply of carbon dioxide to be turned on or off, and in some cases may allow for control over the rate of supply of carbon dioxide. The means for controlling the flow may be operated by a timer switch in order to control the time for which the carbon dioxide is supplied. The timing of supply of carbon dioxide can coincide with the timing of lighting provided for photosynthesis (e.g. lights can be turned off to provide a dark period, as well as saving power), and this avoids wastage of carbon dioxide when photosynthesis is not occurring due to lack of light.

In some examples, the apparatus further comprises means for controlling the temperature of the environment in which the culture vessel is arranged. For example, the environment may be a laboratory or a temperature-controlled growth room. For example, the means for controlling the temperature may be a thermostat and a cooling system for controlling the temperature of the environment (e.g. the growth room). In some examples, the temperature may be between 18 and 27° C. For example, the temperature may be controlled between 18 and 27° C. Preferably, the temperature is around 23° C. Colder temperatures may be used to slow growth rates for production purposes.

In some examples, the inlet pipe is impermeable to carbon dioxide. In some cases, the inlet pipe is made from a material which is impermeable to carbon dioxide. In other cases, the inlet pipe may be coated in a material which is impermeable to carbon dioxide. By “impermeable”, it is preferably meant that carbon dioxide does not diffuse through the material. It is desirable that the inlet pipe does not comprise any holes, pores, or other channels that would permit movement of carbon dioxide through the external surface of the inlet pipe (besides the openings at either end of the pipe). In one example, the inlet pipe may be made from plastic. Preferably, the inlet pipe is made from nylon. In other examples, the inlet pipe may be made from other plastics, such as polyvinyl chloride (PVC), polypropylene (PP), or polyethylene terephthalate (PET).

In some examples, the conduit arrangement comprises one or more conduits for supplying carbon dioxide into the enclosed airspace. For example, the conduit arrangement may comprise one or more conduits, such as one or more pipes, arranged between the source of carbon dioxide and the enclosed airspace. In some examples, the apparatus may comprise a plurality of pipes, such as an inlet pipe connected to the source of carbon dioxide and a second pipe connected to the inlet pipe. Pipes may therefore be connected, e.g. in series, to form the conduit arrangement.

In some examples, the apparatus is configured to provide a sterile supply of carbon dioxide to the enclosed airspace. In some examples, the enclosed airspace is enclosed except for the sterile supply of carbon dioxide. For example, the culture vessel may be sealed except for the sterile supply of carbon dioxide. This allows supply of carbon dioxide without introducing contaminants. The carbon dioxide supply may be sterile by diffusing the carbon dioxide through a barrier permeable to carbon dioxide.

Optionally, the means for supplying carbon dioxide comprises a barrier permeable to carbon dioxide. The barrier is preferably a selective barrier which allows passage of carbon dioxide therethrough. In some examples, other gases present in air such as nitrogen may also be able to permeate through the barrier. For example, the barrier may permit gas exchange. The barrier may be part of the apparatus. In other words, the apparatus may comprise a barrier permeable to carbon dioxide.

Optionally, the barrier is configured to permit passage of carbon dioxide and prevent passage of contaminants into the enclosed airspace. For example, the contaminants may be in the form of micro-organisms, including bacteria and fungi, which are too big to pass through the barrier. For example, the barrier may have passages or pores which permit the passage of small molecules such as carbon dioxide, but prevent passage of large particles such as bacteria. Preferably, the barrier is not permeable to water. This means that carbon dioxide can pass through the barrier, but water cannot.

The presence of the barrier means that the supply of carbon dioxide does not need to be sterile in all cases. In some examples, it is desired to supply carbon dioxide (e.g. in the form of air) to the culture vessel. Avoiding contamination by sterilising the air is costly and complex to achieve, often requiring inconvenient and prohibitively expensive equipment. Instead, the barrier avoids the need for sterilising the carbon dioxide as carbon dioxide can pass through the membrane into the enclosed airspace without contaminants. In other words, the barrier may sterilise the carbon dioxide by permitting the passage of carbon dioxide but excluding contaminants.

In some examples, the barrier may not be permeable to air, meaning that carbon dioxide can be selectively filtered from air, and other gases and contaminants can be prevented from passing through into the enclosed airspace.

In some examples, the barrier is arranged between the source of carbon dioxide and the enclosed airspace. By arranging the barrier in the fluid pathway between the source and the enclosed airspace, the barrier can allow passage of carbon dioxide, while removing unwanted substances such as contaminants, isolating the enclosed airspace from the contaminants. The barrier thus separates the enclosed airspace (in which clean carbon dioxide can diffuse into) from the non-sterile exterior. This can allow extraction of carbon dioxide from a source of carbon dioxide that is not pure, or not sterile.

Optionally, the means for supplying carbon dioxide is configured to supply carbon dioxide through the barrier permeable to carbon dioxide and into the enclosed airspace. In other words, the means for supplying the carbon dioxide may be arranged to supply carbon dioxide to the enclosed airspace through the barrier permeable to carbon dioxide. In some examples, the apparatus is configured to supply carbon dioxide through the barrier permeable to carbon dioxide and into the enclosed airspace. In some examples, the conduit arrangement is configured to supply carbon dioxide through the barrier permeable to carbon dioxide and into the enclosed airspace. For example, the conduit arrangement may be configured to supply carbon dioxide from the source of carbon dioxide, through the barrier, and into the enclosed airspace.

Optionally, the barrier is arranged at least partially in contact with the enclosed airspace. In this way, the barrier permits carbon dioxide to diffuse across the barrier into the enclosed airspace. This also improves the sterility of the apparatus as it avoids the need for further pieces of apparatus such as conduits being required between the barrier and the enclosed airspace. In such cases, this would provide a contamination risk. In other words, the barrier may form an interface with the enclosed airspace. For example, at least one side of the barrier may be arranged within the culture vessel and in contact with the enclosed airspace. The other side of the barrier may be arranged to be in fluid communication with the source of carbon dioxide. Thus, the carbon dioxide can be supplied from the source of carbon dioxide (in some examples, through a conduit arrangement, for example including an inlet pipe), through the barrier, and into the enclosed airspace.

Optionally, the barrier may be arranged at least partially within the culture vessel. For example, the barrier may be arranged at least partially within the enclosed airspace. In some examples, the barrier may be arranged at an edge of the enclosed airspace. For example, the barrier may be arranged within the lid of the culture vessel, or may form the lid. The barrier may therefore form part of an edge of the enclosed airspace, and may define a periphery of the enclosed airspace.

In some examples, the barrier may comprise a membrane. The membrane preferably comprises a flexible material which permits the passage of carbon dioxide. Preferably, the membrane comprises a thickness of material which provides the barrier. For example, the membrane may be made from silicone rubber. The membrane may otherwise be referred to as a filter. Providing a single material permeable to carbon dioxide (e.g. silicone rubber) avoids the need for complex filters.

In one example, the barrier is arranged within the culture vessel, extending across a width of the culture vessel above the culture medium. This defines the enclosed airspace below the barrier. For example, the barrier may be in a planar form extending across the culture vessel. For example, the barrier may be in the form of a membrane. The carbon dioxide can then be supplied into the culture vessel above the barrier, and the carbon dioxide can diffuse through the permeable barrier into the enclosed airspace. The barrier can extend across the entire cross-sectional area of the culture vessel such that carbon dioxide cannot bypass the barrier. As the barrier covers the enclosed airspace, carbon dioxide is forced to pass through the barrier in order to enter the enclosed airspace. The enclosed airspace below the barrier thus contains clean carbon dioxide, while any contaminants remain above the barrier away from contact with the Sphagnum. In some cases, the barrier may be arranged between the inlet pipe and the culture medium. The barrier may be attached to an inner wall of the culture vessel. In some examples, the barrier may form the lid of the culture vessel. For example, the lid may comprise a barrier permeable to carbon dioxide. This allows carbon dioxide to diffuse through the lid and into the enclosed airspace. The barrier thus is arranged partially in contact with the enclosed airspace and forms an interface with the enclosed airspace. In this arrangement, an inlet pipe may not be required.

In some examples, the means for supplying carbon dioxide may be configured to supply carbon dioxide to a volume around the culture vessel. For example, carbon dioxide may be supplied by providing high concentrations of carbon dioxide to a volume around the culture vessel, which can then diffuse through the barrier and into the enclosed airspace. In one example, carbon dioxide levels can be increased in the room. In another example, carbon dioxide can be supplied to a container surrounding the culture vessel. The container may provide a volume in which the carbon dioxide levels can be increased, providing a concentration gradient to permit diffusion into the airspace of the culture vessel. This is preferred because it is safer than increasing the carbon dioxide in the room. The container may be arranged to surround a plurality of culture vessels, so that carbon dioxide can be supplied to a plurality of culture vessels at the same time. The combination of supplying carbon dioxide to a volume (e.g. a container) around the culture vessel and supplying the carbon dioxide from the volume (e.g. the container) through a barrier permeable to carbon dioxide into the enclosed airspace, allows the carbon dioxide to be efficiently supplied without introducing contamination.

In some examples, the barrier has a gas permeability for carbon dioxide of at least 1000 Barrer, preferably at least 2000 Barrer, more preferably at least 3000 Barrer, most preferably around 3250 Barrer, where 1 Barrer is equal to 10⁻¹⁰ cm³_{STP}·cm/(cm²·s·cmHg).

In a preferred example, the barrier is made from silicone. The term “silicone” should preferably be understood to refer to silicone rubber. For example, the barrier may be made from silicone rubber. Silicone is permeable to small molecule gases such as carbon dioxide, while prevents passage of large contaminant particles such as bacteria and fungi. For example, the barrier may comprise dimethylsilicone rubber.

Other gas permeable material may be used for the barrier. For example, the barrier may be made from micropore tape. Preferably, the material is permeable to carbon dioxide while being impermeable to contaminants such as bacteria and fungi, and optionally impermeable to water.

Optionally, the barrier comprises a tube permeable to carbon dioxide. The tube is preferably a conduit for conveying carbon dioxide to the enclosed airspace. In other words, the means for supplying carbon dioxide may comprise a tube permeable to carbon dioxide. In other words, the apparatus may comprise a tube permeable to carbon dioxide. Carbon dioxide is able to flow along the inside of the tube. The tube preferably has a hollow interior for carrying the carbon dioxide, and an external tubular surface containing the interior therein. The tube may have a circular cross-section such that the external surface is generally cylindrical, at least when straight. The tube may be flexible such that it can be bent along its length, and thus the external surface may not be cylindrical in use. In other examples, the tube may be other shapes, and may have other cross-sections, including square or rectangular.

The tube is permeable to carbon dioxide which means that carbon dioxide can permeate through the surface of the tube (i.e. the outer wall of the tube). In one example, this means the tube is made from a material permeable to carbon dioxide. For example, the tube may be made from silicone (i.e. silicone rubber). The tube may be considered to be a barrier because it selectively permits diffusion of carbon dioxide while preventing passage of larger particles and contaminants. The entire tube may be made from the permeable material, or only a portion thereof. For example, the tube may be generally impermeable, but have a permeable section. For example, the permeable section can be a thinner section which is permeable, whereas a thicker section of the tube is impermeable (or much less permeable). However, it is desirable to have a larger surface area permeable in order to increase to potential diffusion rate, and thus it is preferable that the whole tube is permeable.

In some examples, the permeable tube may be a tube which comprises pores for permitting the passage of carbon dioxide. The size of the pores may prevent passage of larger particles such as contaminants and water.

The permeable tube provides a particularly preferable system for supplying carbon dioxide to the airspace because carbon dioxide can easily diffuse out of the tube through the permeable outer surface of the tube through which the carbon dioxide is being conveyed. This means that carbon dioxide can diffuse out across the length of the tube when the whole tube is permeable, and provides a large surface area of diffusion. The diffusion can occur while the carbon dioxide is being transported along the tube.

In this way, the permeable tube can form a barrier arranged between the source of carbon dioxide and the enclosed airspace because the carbon dioxide is forced to diffuse through the outer surface of the tube. The apparatus may be configured to supply carbon dioxide through the tube permeable to carbon dioxide and into the enclosed airspace. For example, the means for supplying carbon dioxide may be configured to supply carbon dioxide through the tube permeable to carbon dioxide and into the enclosed airspace. For instance, carbon dioxide may be supplied through a wall of the tube into the enclosed airspace. The carbon dioxide can diffuse through the permeable wall of the tube.

Optionally, the tube is arranged at least partially in the enclosed airspace. For example, the tube may be arranged at least partially within the culture vessel. In other words, at least part of the tube is arranged in the enclosed airspace. For example, at least part of a surface of the tube is arranged in the enclosed airspace. For example, at least part of a wall of the tube is arranged in the enclosed airspace. By arranging at least part of the tube in the enclosed airspace, carbon dioxide can diffuse through the wall of the tube and into the airspace. This allows carbon dioxide to be supplied to the enclosed airspace, while the permeable tube provides a barrier to prevent contamination. By diffusing through the tube into the enclosed airspace, the concentration of carbon dioxide can be increased in the entire enclosed airspace. The carbon dioxide can then supply the Sphagnum, such as by diffusing into the liquid culture medium. This diffusion can happen over the entire surface area of the boundary between the enclosed airspace and the liquid culture medium. Therefore, this provides a much higher surface area of diffusion than simply arranging the tube within the liquid culture medium. In some examples where the culture medium is a liquid culture medium, the tube is preferably not arranged within the liquid culture medium.

In some examples, the tube is closed so that carbon dioxide is forced to diffuse through the tube. In other words, preferably the tube does not have an open end arranged in the enclosed airspace. This ensures that carbon dioxide is forced to pass through the permeable surface of the tube.

In some examples, the tube is arranged such that both ends of the tube are arranged outside the enclosed airspace and part of the tube between the ends is arranged within the enclosed airspace. This forms a loop portion of tube within the enclosed airspace. Because both ends of the tube are arranged outside the enclosed airspace, risk of contamination at the connection with other pipes or conduits is minimised, while ease of use is improved. Moreover, the carbon dioxide is forced through the wall of the tube at the loop portion. In other words, the tube is arranged to extend through an inlet of the culture vessel into the enclosed airspace, through the enclosed airspace, and through an outlet of the culture vessel out of the enclosed airspace. In one example, the tube can be fed into and out of the enclosed airspace so that a loop of tube is provided in the enclosed airspace through which carbon dioxide can diffuse.

The barrier defines a region between the source of carbon dioxide and the barrier. For example, in the case of a barrier extending across a width of the culture vessel, a region is defined above the barrier in fluid communication with the source of carbon dioxide. This region includes the remaining volume of the culture vessel above the barrier which is separated from the enclosed airspace by the barrier, and any volume of the inlet pipe connected to the source of carbon dioxide. In the example of a volume or container around the culture vessel, the region includes the volume of the container around the culture vessel. In the example of the permeable tube, the region is defined within the volume of the tube and up to the source of carbon dioxide. This region is a volume in which carbon dioxide is supplied from the source of carbon dioxide up to the barrier. This region preferably contains a concentration of carbon dioxide which is higher than the atmospheric concentration. In other words, carbon dioxide is supplied to the barrier at a higher concentration than if the barrier were open to the environment. This improves the diffusion rate of carbon dioxide and provides a sufficient carbon supply for desired growth.

Preferably, the region also comprises a pressure which is greater than atmospheric pressure. This promotes the supply of carbon dioxide through the barrier and into the enclosed airspace, improving the supply of carbon dioxide for growth.

In some examples, the tube has a length of more than 3 cm. In some examples, the tube has a length of at least 5 cm, preferably at 10 cm, more preferably about 15 cm. For example, the tube may extend at least 3 cm in the enclosed airspace, preferably at least 5 cm, more preferably about 10 cm. Most preferably, the tube has a length of around 15 cm, around 10 cm of which is within the enclosed airspace. This is particularly preferable where the culture vessels is a 2 L or 5 L container. The length of the tube, especially the length of the tube arranged in the airspace can be chosen to determine the diffusion rate into the airspace. A longer tube provides a greater surface area for diffusion.

In some examples, the tube has an inner diameter of at least 1 mm, preferably at least 2 mm, more preferably around 3 mm. In some cases, the tube may have an inner diameter of between 1 mm and 6 mm, preferably between 2 mm and 5 mm. Most preferably, the tube has an inner diameter of about 3 mm. The diameter determines the volume of carbon dioxide supplied and the available surface area for diffusion. The larger the diameter the more surface area provided, but this also causes a drop in pressure. With a larger diameter, the connections to other tubes becomes more difficult, and the cost of the tube increases significantly. Therefore, an optimum inner diameter of around 3 mm is desired.

In some examples, the tube has a thickness of at least 0.5 mm, preferably at least 0.75 mm, more preferably around 1 mm. In some cases, the tube may have a thickness of between 0.5 mm and 2 mm, preferably between 0.75 mm and 1.5 mm. Most preferably, the tube has a thickness of around 1 mm. The thickness will affect the structural integrity of the tube, and also the ability to diffuse carbon dioxide into the enclosed airspace. The thicker the tube, the more rigid the tube, so it is desirable to avoid providing a rigid tube that is not deformable to form an appropriate seal e.g. with the inlet pipe. However, if the tube is too thin, then the shape of the tube will not be well maintained, and the tube may collapse in on itself. The thinner the tube, the higher the rate of diffusion, so it is desirable to provide a thin tube which still provides the necessary rigidity. Therefore, an optimum thickness of around 1 mm is desired.

It is particularly preferable that the inner diameter is chosen considering the thickness, because this permits suitable diffusion. In some cases, the tube has an inner diameter of between 1 mm and 6 mm and a thickness of between 0.5 mm and 2 mm, preferably the tube has an inner diameter of between 2 mm and 5 mm and a thickness of between 0.75 mm and 1.5 mm. Most preferably, the tube has an inner diameter of around 3 mm, and the tube has a thickness of around 1 mm. This provides the optimum diffusion and volume supply of carbon dioxide, while providing the desired structural features.

Optionally, a first end of the tube may be connected to the inlet pipe. The tube can be connected to the inlet pipe such that the tube provides a fluid pathway for carbon dioxide from the inlet pipe to the enclosed airspace of the culture vessel. This may be the same inlet pipe as directly connected to the source of carbon dioxide. In other words, a first end of the inlet pipe may be connected to the source of carbon dioxide and a second end connected to the permeable tube. In other examples, the tube may be connected to a pipe of the conduit arrangement. The pipe may in turn be connected to another pipe which is connected to the source of carbon dioxide. In one example, a first end of the tube is connected to the inlet pipe by attaching the tube over the inlet pipe. In this example, the tube has an internal diameter approximately equal (or slightly less than, if deformable) to an external diameter of the inlet pipe such that the tube can fit over the inlet pipe. The tube can then form a friction fit over the end of the inlet pipe forming a seal. In other examples, the tube can friction fit inside the inlet pipe in which case an external diameter of the tube is approximately equal (or slightly larger, if deformable) to an internal diameter of the inlet pipe. In yet other examples, the tube can be connected to the inlet pipe via any form of pipe connector or pipe fitting, in which case the diameters can vary. Silicone rubber has been found to be a particularly effective material for the tube which is deformable to allow the formation of a seal with the inlet pipe to inhibit leakage.

In some examples, the inlet pipe may extend through an inlet of the culture vessel. For example, the inlet may be referred to as an inlet hole. In some examples, the culture vessel comprises an inlet arranged in a wall of the culture vessel, preferably in an upper surface. In one example, the culture vessel comprises a lid. For example, the lid may be airtight such that the airspace remains enclosed when the lid is closed. In some cases, the lid is removable. In cases where a lid is provided, the inlet may be arranged in the lid. This allows access to the airspace above the culture medium arranged at the upper end of the culture vessel. Otherwise, the inlet may be arranged in a side wall of the culture vessel above the culture medium. The tube may then be attached to the end of the inlet pipe. In this manner, the tube is arranged entirely inside the culture vessel, so that carbon dioxide is not released outside the culture vessel.

In some examples, an open end of the inlet pipe can be arranged in the enclosed airspace and is configured to release carbon dioxide into the enclosed airspace. In some examples, an open end of the inlet pipe can be arranged in the culture vessel and is configured to release carbon dioxide into the culture vessel. In other words, rather than the inlet pipe connecting to a permeable tube, the inlet pipe is connected to the culture vessel and directly supplies carbon dioxide into the interior of the culture vessel. In such cases, a barrier may be arranged so that carbon dioxide released from the open end of the inlet pipe can permeate therethrough and into the enclosed airspace. For example, the barrier may be in the form of a membrane. In another example, the barrier may be in the form of a permeable cap over the end of the open end of the inlet pipe.

In some examples, the tube is arranged to extend through an inlet of the culture vessel. For example, the inlet may be referred to as an inlet hole. In such examples, the inlet pipe can remain external of the culture vessel, and the tube extends through an inlet of the culture vessel. In this case, the tube is partially arranged outside the culture vessel. This keeps the inlet pipe outside which means it does not need to be sterile. It is preferable to keep the length of the tube outside the culture vessel to a minimum to avoid carbon dioxide losses external of the culture vessel, and also avoid raising the carbon dioxide concentration of the room in which the culture vessel is arranged. Providing the connection external to the culture vessel also provides for easier and more convenient connection and disconnection of the inlet pipe from the outlet. As such, it is preferable to provide the tube extending through the inlet and have the connection external to the culture vessel rather than the inlet pipe extending through the inlet and have the connection internal. In some cases where the tube extends through the inlet, the tube may be directly connected to the source of carbon dioxide. In this situation, there may not be a separate inlet pipe as such because the section of the tube connected to the source of carbon dioxide functionally forms the inlet pipe. In other cases, the tube may pass through the inlet and connect to the inlet pipe or other part of the conduit arrangement. The first end of the tube may be aligned with the inlet or may protrude such that a portion of the tube is arranged externally of the culture vessel.

The tube may have an outer diameter which is greater than the diameter of the inlet. In this way, the tube is configured to deform slightly to fit through the inlet and form a seal. This maintains the carbon dioxide levels within the culture vessel, and prevent ingress of contamination. Providing the tube made from silicone rubber has been found to be effective. In other examples, a seal may be provided by use of a separate deformable ring, or a sealant or adhesive may be used.

In some examples, the tube forms the inlet pipe, and a separate inlet pipe is not provided. In some cases, the tube can have a permeable part as described above for releasing carbon dioxide into the enclosed airspace, and also has an impermeable part which can act as the inlet pipe. For example, the impermeable part can connect the permeable part with the source of carbon dioxide.

In some examples, the tube is arranged to extend through an outlet of the culture vessel. For example, the outlet may be referred to as an outlet hole. In some examples, the culture vessel comprises an outlet arranged in a wall of the culture vessel, preferably in an upper surface. The outlet may have similar features described with reference to the inlet. In some cases, the outlet is arranged in the lid. Otherwise, the outlet may be arranged in a side wall of the culture vessel above the culture medium. In particular, the second end of the tube, opposite to the first end which is connected to the inlet pipe, may be arranged through the outlet. The second end of the tube may be arranged through the outlet while the first end of the tube may be arranged through the inlet. The tube may be arranged so that the first end and the second end are each arranged externally of the culture vessel, and a portion of the tube between the ends is arranged within the culture vessel (within the enclosed airspace). This means the tube defines a continuous conduit from the inlet pipe to the outlet, meaning that carbon dioxide cannot be released into the culture vessel except by diffusion through the wall of the permeable tube. In some examples, the tube forms a continuous conduit between the inlet and the outlet. This means the only path into the enclosed airspace is through the permeable walls of the tube. The carbon dioxide is also routed into the culture vessel within the tube, and also routed out. This means carbon dioxide that does not diffuse (including other gases and contaminants) is removed from the tube and can be recycled, such as by cycling through the conduit arrangement again or supplied to another culture vessel. Also, in this way, a series of culture vessels can be supplied with carbon dioxide by using a continuous conduit, as will be described in more detail below. The second end of the tube may be aligned with the outlet or may protrude such that a portion of the tube is arranged externally of the culture vessel.

Optionally, the apparatus further comprises an outlet pipe connected to a second end of the tube. In some examples, the apparatus comprises an outlet pipe. In some examples, the conduit arrangement may comprise an outlet pipe. The outlet pipe may comprise one or more features described in relation to the inlet pipe. For example, the outlet pipe may be impermeable to carbon dioxide. The second end of the tube is at the opposite end than the first end of the tube which can be connected to the inlet pipe. In other words, the tube extends between the first end and the second end. Thus, the tube forms a duct from the inlet pipe to the outlet pipe. This provides a continuous conduit from the inlet pipe to the outlet pipe. This means carbon dioxide cannot enter the enclosed airspace except by diffusion through the tube.

The outlet pipe may be provided for supplying carbon dioxide to a second culture vessel. In other words, carbon dioxide which is not diffused through the wall of the permeable tube can be conveyed through the outlet pipe and into another culture vessel, such as through a second permeable tube.

In some examples, the outlet pipe may extend through the outlet of the culture vessel. The tube may then be attached to the end of the outlet pipe. In this manner, the tube is arranged inside the culture vessel, so that carbon dioxide is not released outside the culture vessel. As above, it is preferable to provide the tube extending through the outlet and have the connection to the outlet pipe external to the culture vessel rather than the outlet pipe extending through the outlet and providing the connection internal to the culture vessel. The tube may have an outer diameter which is greater than the diameter of the outlet. In this way, the tube is configured to deform slightly to fit through the outlet and form a seal. This maintains the carbon dioxide levels within the culture vessel, and prevent ingress of contamination. Providing the tube made from silicone rubber has been found to be effective. In other examples, a seal may be provided by use of a separate deformable ring, or a sealant or adhesive may be used.

In some examples, the tube forms the outlet pipe, and a separate outlet pipe is not provided. In some cases, the tube can have a permeable part as described above for releasing carbon dioxide into the enclosed airspace, and also has an impermeable part which can act as the outlet pipe. For example, the impermeable part can connect the permeable part with the inlet pipe or the tube of the adjoining culture vessel. In other cases, the tube may pass through the outlet and connect to the outlet pipe or other part of the conduit arrangement. The second end of the tube may be aligned with the outlet or may protrude such that a portion of the tube is arranged externally of the culture vessel.

In some examples, a separate inlet pipe and outlet pipe are not provided. In such cases, the tube can be arranged to extend between adjoining culture vessels. For example, the tube can be threaded in through an inlet and out through an outlet of the first culture vessel, and then threaded in through an inlet and out through an outlet of the second culture vessel, continuously connecting the first and second culture vessels. It can be preferable to reduce the length of the tube external to the culture vessels to reduce losses of carbon dioxide which reduces efficiency and can be dangerous in large quantities. This can be done by positioning the outlet of the first culture vessel close to the inlet of the second culture vessel. This can further be improved by providing an impermeable part of the tube between the culture vessels, such as providing a thicker wall or an impermeable coating or sleeve. However, as explained above, it is preferable to provide a permeable tube and an impermeable pipe (e.g. inlet and/or outlet pipes) connecting the tubes of adjoining culture vessels because this provides a simpler, cheaper, and safer solution.

In some examples, an open end of the outlet pipe can be arranged in the enclosed airspace and is configured to allow removal of gas from the airspace. For example, a pump may be used to ensure even flow of gas through the inlet and the outlet. In such cases, a permeable tube may not be provided. This also ensures that the diffusion of carbon dioxide through the barrier into the enclosed airspace is encouraged by ensuring the pressure in the culture vessel is not too high to inhibit potential diffusion.

The inlet and the outlet can be sealed around the inlet pipe, the tube, and/or the outlet pipe, as applicable. The inlet and outlet can be substantially sealed by ensuring the conduit passing therethrough is suitably sized to seal the hole. In some cases, the tube can be deformable so it can be inserted through the inlet and/or the outlet to form a seal.

In some examples, the barrier extends over the open end of the inlet pipe. For example, the barrier may be in the form of a cap arranged between the source of carbon dioxide and the enclosed airspace. In other words, the open end of the inlet pipe may be closed by a barrier. Carbon dioxide is thus forced to diffuse across the barrier, allowing for control of contaminants. This can provide a very simple arrangement without any complex filters, but the area of diffusion is limited by the size of the end of the pipe. In contrast, when a permeable tube is used, the available diffusion area is the entire surface area of the tube, rather than just the area of the end of the inlet pipe, allowing for much higher diffusion rates.

In some examples, the permeable tube is arranged through the lid of the culture vessel (e.g. at the inlet and the outlet). This can provide a lid which is removable with the tube. This allows the tube to easily be sterilised and can be sterilised with the lid of the culture vessel. In some examples, the tube is arranged through an inlet and outlet which is arranged towards a side of the culture vessel in the lid. In other words, the inlet and outlet are arranged adjacent a rim of the lid. This means that if culture vessels are stacked on top of each other, the culture vessel does not interfere with the permeable tube below. In other words, the inlet and the outlet are not covered by the base of the upper culture vessel. The tube is still accessible at the side of the upper culture vessel. To achieve this, the diameter of the base of the culture vessel may be smaller than a diameter at the rim (at the lid), as the side is slightly tapered. This also allows connection and disconnection of the tube to the inlet pipe and/or outlet pipe without unstacking.

In some examples, the means for supplying carbon dioxide is configured to provide a concentration of carbon dioxide in the enclosed airspace of at least 1,000 ppm, preferably at least 2,500 ppm, more preferably at least 5,000 ppm, even more preferably at least 10,000 ppm, still more preferably at least 20,000 ppm, yet still more preferably at least 30,000 ppm. In some cases, the concentration of carbon dioxide in the enclosed airspace may be up to 50,000 ppm. For example, preferably the concentration of carbon dioxide in the enclosed airspace is between 2,000 ppm and 50,000 ppm, more preferably between 5,000 ppm and 50,000 ppm.

In some examples, the carbon dioxide may be supplied to ensure that the outlet pipe bubbles when placed into a liquid. For example, even in cases where a plurality of culture vessels are connected together in series (e.g. up to 96 culture vessels), the final outlet pipe may be placed into a liquid, and if bubbles are formed, then the flow rate can be considered to be sufficient. In some examples, the flow rate can be controlled to ensure bubbles form.

In some examples, the culture medium comprises nutrients for facilitating growth of Sphagnum. This can improve the growth rates.

In some examples, the culture medium comprises nutrients.

In some examples, the culture medium comprises nutrients comprising nitrogen, phosphorus, potassium, calcium, magnesium, sodium, manganese, copper, zinc, sulfur, boron, iron, molybdenum, and/or chlorine. In some examples, the culture medium comprises nutrients comprising nitrogen, phosphorus, potassium, calcium, magnesium, sodium, manganese, copper, zinc, sulfur, boron, iron, molybdenum, chlorine, cobalt, and/or iodine.

Optionally, the culture medium may comprise nitrogen, phosphorus, potassium, calcium, magnesium, sodium, manganese, copper, zinc, sulfur, boron, iron, molybdenum, chlorine, cobalt, and iodine.

In some examples, the culture medium comprises at least 18.05 mg per L of nitrogen. In some examples, the culture medium comprises between 18.05 mg and 103.98 mg per L of nitrogen. Preferably, the culture medium comprises at least 40 mg per L of nitrogen. More preferably, the culture medium comprises between 40 mg and 55 mg per L of nitrogen. In some examples, the culture medium comprises less than 103.98 mg per L of nitrogen. For example, nitrogen may be present in nitrite, nitrate, and/or ammonium form. For example, nitrogen may be provided by (e.g. disodium) EDTA, (e.g. ferrous sodium) DTPA, ammonium nitrate (NH₄NO₃), and/or calcium nitrate (Ca(NO₃)₂·H₂O).

In some examples, the culture medium comprises at least 9.12 mg per L of phosphorus. In some examples, the culture medium comprises at least 10.99 mg per L of phosphorus. In some examples, the culture medium comprises between 10.99 mg and 54.02 mg per L of phosphorus. Preferably, the culture medium comprises at least 5 mg per L of phosphorus. More preferably, the culture medium comprises between 5 mg and 15 mg per L of phosphorus. For example, phosphorus may be provided by potassium (dihydrogen) phosphate (KH₂PO₄).

In some examples, the culture medium comprises at least 66.84 mg per L of potassium. In some examples, the culture medium comprises between 66.84 mg and 151.10 mg per L of potassium. Preferably, the culture medium comprises at least 120 mg per L of potassium. More preferably, the culture medium comprises between 120 mg and 130 mg per L of potassium. For example, potassium may be provided by potassium (dihydrogen) phosphate (KH₂PO₄), potassium sulfate (K₂SO₄), and/or potassium iodide (KI).

In some examples, the culture medium comprises at least 1.17 mg per L of calcium. In some examples, the culture medium comprises between 1.17 mg and 36.96 mg per L of calcium. Preferably, the culture medium comprises at least 25 mg per L of calcium. More preferably, the culture medium comprises between 25 mg and 35 mg per L of calcium. For example, calcium may be provided by calcium nitrate (Ca(NO₃)₂·H₂O) and/or calcium chloride dihydrate (CaCl₂·2H₂O).

In some examples, the culture medium comprises at least 0.33 mg per L of magnesium. In some examples, the culture medium comprises between 0.33 mg and 13.17 mg per L of magnesium. Preferably, the culture medium comprises at least 5 mg per L of magnesium. More preferably, the culture medium comprises between 5 mg and 15 mg per L of magnesium. For example, magnesium may be provided by magnesium sulfate (MgSO₄·7H₂O)

In some examples, the culture medium comprises at least 1.32 mg per L of sodium. In some examples, the culture medium comprises at least 2.51 mg per L of sodium. In some examples, the culture medium comprises between 2.51 mg and 53.47 mg per L of sodium. In some examples, the culture medium comprises at least 0.1 mg per L of sodium. Preferably, the culture medium comprises at least 1 mg per L of sodium. More preferably, the culture medium comprises between 1 mg and 10 mg per L of sodium. For example, sodium may be provided by disodium EDTA, (e.g. ferrous) sodium DTPA, and/or sodium molybdate (Na₂MoO₄·2H₂O).

In some examples, the culture medium comprises at least 3 mg per L of manganese. In some examples, the culture medium comprises at least 5.49 mg per L of manganese. In some examples, the culture medium comprises at least 0.21 mg per L of manganese. In some examples, the culture medium comprises between 0.21 mg and 1.94 mg per L of manganese. Preferably, the culture medium comprises at least 1 mg per L of manganese. More preferably, the culture medium comprises between 1 mg and 10 mg per L of manganese. For example, manganese may be provided by manganese sulfate (MnSO₄·4H₂O).

In some examples, the culture medium comprises at least 0.01 mg per L of copper. In some examples, the culture medium comprises at least 0.09 mg per L of copper. In some examples, the culture medium comprises between 0.09 mg and 0.25 mg per L of copper. Preferably, the culture medium comprises at least 0.01 mg per L of copper. More preferably, the culture medium comprises between 0.01 mg and 0.25 mg per L of copper. For example, copper may be provided by copper sulfate (CuSO₄·5H₂O).

In some examples, the culture medium comprises at least 0.37 mg per L of zinc. In some examples, the culture medium comprises between 0.37 mg and 1.56 mg per L of zinc. Preferably, the culture medium comprises at least 1 mg per L of zinc. More preferably, the culture medium comprises between 1 mg and 2 mg per L of zinc. For example, zinc may be provided by zinc sulfate (ZnSO₄O·7H₂O).

In some examples, the culture medium comprises at least 4.30 mg per L of sulfur. In some examples, the culture medium comprises between 4.30 mg and 65.59 mg per L of sulfur. In some examples, the culture medium comprises at least 40 mg per L of sulfur. Preferably, the culture medium comprises at least 60 mg per L of sulfur. More preferably, the culture medium comprises between 60 mg and 70 mg per L of sulfur. For example, sulfur may be provided by zinc sulfate (ZnSO₄·7H₂O).

In some examples, the culture medium comprises at least 0.14 mg per L of boron. In some examples, the culture medium comprises between 0.14 mg and 1.02 mg per L of boron. In some examples, the culture medium comprises at least 0.5 mg per L of boron. Preferably, the culture medium comprises at least 0.6 mg per L of boron. More preferably, the culture medium comprises between 0.6 mg and 1.5 mg per L of boron. For example, boron may be provided by boric acid (H₃BO₃).

In some examples, the culture medium comprises at least 0.31 mg per L of iron. In some examples, the culture medium comprises between 0.31 mg and 9.15 mg per L of iron. In some examples, the culture medium comprises at least 3 mg per L of iron. Preferably, the culture medium comprises at least 1 mg per L of iron. More preferably, the culture medium comprises between 1 mg and 10 mg per L of iron. For example, iron may be provided by ferrous sulfate (FeSO₄·7H₂O) and/or ferrous (e.g. sodium) DTPA.

In some examples, the culture medium comprises at least 0.01 mg per L of molybdenum. In some examples, the culture medium comprises between 0.01 mg and 0.15 mg per L of molybdenum. Preferably, the culture medium comprises at least 0.1 mg per L of molybdenum. More preferably, the culture medium comprises between 0.1 mg and 0.15 mg per L of molybdenum. For example, molybdenum may be provided by sodium molybdate (Na₂MoO₄·2H₂O).

In some examples, the culture medium comprises at least 0.16 mg per L of chlorine. In some examples, the culture medium comprises between 0.16 mg and 97.64 mg per L of chlorine. Preferably, the culture medium comprises at least 10 mg per L of chlorine. More preferably, the culture medium comprises between 10 mg and 25 mg per L of chlorine. For example, chlorine may be provided by calcium chloride dihydrate (CaCl₂·2H₂O) and/or cobalt chloride (CoCl₂·6H₂O).

In some examples, the culture medium comprises at least 0.006 mg per L of cobalt. In some examples, the culture medium comprises at least 0.001 mg per L of cobalt. For example, chlorine may be provided by cobalt chloride (CoCl₂·6H₂O).

In some examples, the culture medium comprises at least 0.64 mg per L of iodine. In some examples, the culture medium comprises at least 0.10 mg per L of iodine. For example, chlorine may be provided by potassium iodide (KI).

In some examples, the culture medium comprises:
a) at least 18.05 mg per L of nitrogen;
b) at least 10.99 mg per L of phosphorus;
c) at least 66.84 mg per L of potassium;
d) at least 1.17 mg per L of calcium;
e) at least 0.33 mg per L of magnesium;
f) at least 2.51 mg per L of sodium;
g) at least 0.21 mg per L of manganese;
h) at least 0.09 mg per L of copper;
i) at least 0.37 mg per L of zinc;
j) at least 4.30 mg per L of sulfur;
k) at least 0.14 mg per L of boron;
l) at least 0.31 mg per L of iron;
m) at least 0.01 mg per L of molybdenum; and/or
n) at least 0.16 mg per L of chlorine.

The culture medium may further comprise at least 0.006 mg per L of cobalt and/or at least 0.64 mg per L of iodine.

In a preferred example, the culture medium comprises:
a) at least 40 mg per L of nitrogen;
b) at least 5 mg per L of phosphorus;
c) at least 120 mg per L of potassium;
d) at least 25 mg per L of calcium;
e) at least 5 mg per L of magnesium;
f) at least 1 mg per L of sodium;
g) at least 1 mg per L of manganese;
h) at least 0.01 mg per L of copper;
i) at least 1 mg per L of zinc;
j) at least 60 mg per L of sulfur;
k) at least 0.6 mg per L of boron;
l) at least 1 mg per L of iron;
m) at least 0.1 mg per L of molybdenum; and/or
n) at least 10 mg per L of chlorine.

The culture medium may further comprise at least 0.006 mg per L of cobalt and/or at least 0.64 mg per L of iodine.

In a more preferred example, the culture medium comprises:
a) between 40 mg and 55 mg per L of nitrogen;
b) between 5 mg and 15 mg per L of phosphorus;
c) between 120 mg and 130 mg per L of potassium;
d) between 25 mg and 35 mg per L of calcium;
e) between 5 mg and 15 mg per L of magnesium;
f) between 1 mg and 10 mg per L of sodium;
g) between 1 mg and 10 mg per L of manganese;
h) between 0.01 mg and 0.25 mg per L of copper;
i) between 1 mg and 2 mg per L of zinc;
j) between 60 mg and 70 mg per L of sulfur;
k) between 0.6 mg and 1.5 mg per L of boron;
l) between 1 mg and 10 mg per L of iron;
m) between 0.1 mg and 0.15 mg per L of molybdenum; and/or
n) between 10 mg and 25 mg per L of chlorine.

The culture medium may further comprise at least 0.006 mg per L of cobalt and/or at least 0.64 mg per L of iodine.

Because the Sphagnum is cultured with supply of carbon dioxide instead of sugar, a culture medium with relatively high nutrient content can be used. By mitigating contamination risk by using carbon dioxide instead of sugar, higher nutrient levels can be used, which permits faster growth. It has also been found that when Sphagnum can be grown substantially axenically, high nutrient levels promote growth. Conventional methods provide little or no nutrients to Sphagnum as it is widely understood that Sphagnum grows in environments with little or no nutrient supply. However, by supplying carbon dioxide and culturing without sugar, growth of Sphagnum can be improved by supplying with high levels of nutrients without causing significant contamination.

Optionally, the culture medium does not comprise sugar. For example, the culture medium does not have sugar, such as sucrose or glucose, added to it. This avoids contaminant growth as described herein. Instead, carbon is provided in the form of carbon dioxide. In other words, the culture medium is sugar-free or sugarless.

Optionally, the culture medium comprises a liquid culture medium. The liquid medium is preferably a liquid comprising water. In other words, the liquid medium may be an aqueous solution. This is preferable because the supply of nutrients can be increased compared to a solid culture medium because the nutrients can be supplied over the surface area of the Sphagnum in contact with the liquid culture medium. The liquid medium is also able to uptake carbon dioxide from the enclosed airspace at a faster rate.

Optionally, the apparatus further comprises means for stirring the liquid culture medium. The means for stirring the liquid medium is preferably configured to stir the liquid medium. For example, the means for stirring the liquid medium may comprise a device configured to stir the liquid medium. For example, optionally the apparatus further comprises a stirrer configured to stir the liquid medium. Preferably, the liquid medium is stirred such that the liquid medium at an upper surface of the liquid medium at the boundary to the enclosed airspace is displaced. In other words, the liquid medium is mixed. The stirring moves the liquid around so that uptake of carbon dioxide can be increased. This moves liquid with a lower concentration of carbon dioxide to the boundary with the enclosed airspace, meaning that the concentration gradient can be maximised, and diffusion rates can be improved, avoiding saturation. This is particularly advantageous when carbon dioxide is supplied to the enclosed airspace, because the concentration of carbon dioxide can be increased in the enclosed airspace, and this in turn can diffuse into the liquid medium for use by the Sphagnum. The stirring thereby promotes uptake of carbon dioxide by the liquid medium from the enclosed airspace, increasing the supply of carbon dioxide to the Sphagnum.

In some examples, the means for stirring may comprise a stirring mechanism. For example, the stirring mechanism may comprise a mixer configured to rotate to stir the liquid medium. For example, the mixer may be a rotating stirring device arranged within the culture vessel. In another example, the stirrer may be operated by a magnetic connection from outside of the culture vessel. In other cases, the bottom of the culture vessel may comprise a mixer mounted therein.

Optionally, the means for stirring is arranged externally of the culture vessel. In other words, the means for stirring is not arranged within the culture vessel. For example, the external stirring means may comprise an external agitator to tilt, vibrate, shake, or otherwise agitate the culture vessel to stir the liquid medium. It is preferable that such agitation or stirring by the mixer does not pulverise the Sphagnum or break it into small pieces. Stirring the medium from outside has the advantage that it does not interfere with the Sphagnum within the culture vessel. Internal mixers have been found to lead to Sphagnum tangling on the mixer. Preferably, the stirring is performed when the Sphagnum is in the gametophore (or gametophyte, or adult) stage of growth. This should be distinguished from the protoplast or protonema stage. This encourages uptake of carbon dioxide without disturbing the Sphagnum.

Optionally, the means for stirring comprises a heat source configured to apply a temperature differential in the culture vessel. In other words, the apparatus may comprise a heat source configured to apply a temperature differential in the culture vessel to stir the liquid culture medium. For example, the temperature differential may be across a width of the culture vessel. The temperature differential can cause a convection current to flow in the liquid medium to stir the liquid medium. In some examples, the temperature differential is at least 1° C., preferably at least 2° C., more preferably at least 2.5° C., even more preferably at least 3° C.

Providing a heat source has been found to be a simple, cheap, and effective means for stirring. External agitators involve complex and expensive apparatus which requires moving the entire culture vessel, which is not feasible on a large scale, and may cause damage to the Sphagnum if vigorous. These also require significant cost, time, and energy to set up and operate. In contrast, using a heat source can stir the liquid without moving the culture vessel and agitating the Sphagnum.

In some examples, the heat source may be arranged at one side of the culture vessel. For example, the heat source is arranged at one side of the culture vessel and not at the other side. In other words, the heat source is arranged at only one side. In cases where the culture vessel is generally cylindrical, the “side” refers to a point on the circumference, and the “other side” refers to a point on the circumference which is opposite and separated by a diameter of the culture vessel. This ensures that the temperature at the near side of the culture vessel is higher than at the other side, so that a temperature differential can be provided. In some examples, a heat source may be arranged around no more than half of the perimeter of the culture vessel. For example, multiple heat sources may be arranged at locations towards one side, extending around less than half of the perimeter (e.g. the circumference) of the culture vessel. This allows a temperature differential to form. In some cases, a heat source may be arranged at the opposite side, but is further away than the heat source at the near side. In such cases, the heat source is configured to apply a temperature differential as the near-side heat source provides more of a heating effect than the far heat source which is further away. In other words, there is a heat source arranged closer to one side of the culture vessel. Preferably, there is not another heat source arranged at the opposite side of the culture vessel at the same distance or closer.

In some examples, the heat source is arranged less than 20 cm from the side of the culture vessel. In some examples, the heat source is arranged less than 10 cm from the side of the culture vessel. For example, the heat source may be arranged at a distance less than a width of the culture vessel from the side of the culture vessel.

Optionally, the heat source comprises a light source. As light sources are typically used for culturing plants to provide light for photosynthesis (and equally are beneficial for culturing Sphagnum), the present inventors have found that wasted heat energy from light sources may be harnessed and used as a means for stirring as described herein. In other words, by suitable arrangement of light sources, heat from these can be used to apply a temperature differential across the culture vessel to stir the liquid medium and increase uptake of the carbon dioxide from the enclosed airspace. This has been found to be a particularly preferred embodiment, and provides a very effective, efficient, and convenient arrangement.

The light source may be the same light source as used to provide light for photosynthesis. In some examples, the means for stirring may be provided by a dedicated heater alongside a light source for providing light for photosynthesis.

In some examples, the light source is configured to emit white light. This provides light over a spectrum useful for photosynthesis. In some examples, the light source is configured to emit blue and red light. In some examples, the light source is configured to emit light within a photosynthetically active radiation range (e.g. 400 nm to 700 nm).

In some examples, the light source is a fluorescent light. In other examples, the light source may be a halogen light or an incandescent light. In some examples, the light may comprise a light emitting diode (LED). For the purpose of supplying light, it is preferable for the light source to be electrically efficient in terms of producing a high light power output for a given electrical power input, so that the power wasted in heat energy is minimized. This reduces ongoing costs due to electrical power. LEDs are generally more power efficient, but can have high upfront costs to provide the desired light output. Moreover, as disclosed herein, it can be desirable to provide some heat energy to provide the stirring effect. The inventors have surprisingly found that using a light source which has a relatively high efficiency to provide sufficient light and avoid high electrical power costs, while providing sufficient heat energy to stir the liquid medium, results in an improved apparatus for culturing Sphagnum. In other words, a separate heat source is not required, as the wasted heat from the light can be harnessed. It has been found that fluorescent lights are particularly well-suited for this. Although LEDs are becoming more commercially viable, the increased efficiency can actually lead to a drop in the heat energy that can be used, and thus may be less preferable than the use of fluorescent lights. However, in some cases where the cost of electricity is more important, LEDs may be used such as in the form of a tube.

In some examples, the fluorescent light comprises a fluorescent tube. For example, the fluorescent tube may be a white fluorescent tube. For example, the fluorescent tube may have an electrical power of 36 W. This provides the appropriate lighting amount, while avoiding wasted power. Higher power lights may be used, but this has little added benefit to the growth, while leading to significantly higher electrical power costs. Fluorescent tubes have been found to emit light at a desirable frequency and intensity for culturing Sphagnum, and are cost-effective for setup costs and ongoing costs. Fluorescent tubes also emit light in a 360° angle around the longitudinal axis of the length of the tube. This allows full use of the light by surrounding the fluorescent tube with culture vessels.

In some examples, the light source is an LED tube. An LED tube typically has a row of LEDs arranged along the length of the tube, with two rows back-to-back to provide a substantially 360° angle of emission of light.

In some examples, the light source is arranged vertically at one side of the culture vessel. In other words, the light source is arranged to extend in a direction substantially parallel to the longitudinal axis of the culture vessel.

In some examples, the light source is configured to supply a light intensity of at least 25 μmol m⁻²s⁻¹ photosynthetically active radiation (PAR) (i.e. wavelength of 400 to 700 nm), preferably at least 50 μmol m⁻² s⁻¹ PAR. In some examples, the light source is configured to supply a light of intensity of between 200 μmol m⁻² s⁻¹ PAR and 300 μmol m⁻² s⁻¹ PAR. However, this has been found to have a small effect on growth rate for a substantial increase in cost of electrical power. Therefore, it is desirable to provide a light intensity of between 25 and 150 μmol m⁻² s⁻¹ PAR, preferably between 50 and 125 μmol m⁻² s⁻¹ PAR, more preferably between 50 and 110 μmol m⁻² s⁻¹ PAR.

In some examples, an interior of the culture vessel is substantially sterile. For example, an interior of the culture vessel may be sterilised. The tube may also be sterilised if this is arranged within the enclosed airspace. This means that other components such as the inlet pipe do not need to be sterilised because the carbon dioxide is sterilised by passing through the wall of the tube.

In some examples, the culture medium is sterile.

In some examples, the culture vessel is made from plastic. For example, the culture vessel may be made from plastic, such as polypropylene. This allows for easy sterilisation and cleaning, while reducing cost. In other examples, other plastics such as polyvinyl chloride may be used. In other examples, the culture vessel may be made from glass, but this can be more costly and heavier.

In some examples, walls of the culture vessel are transparent. For example, the culture vessel may be made from a transparent plastic. This allows light to be absorbed by the Sphagnum therein. In other examples, the culture vessel may have a window for passage of light.

In some examples, the culture vessel has a volume between 0.1 L and 100 L. For the avoidance of doubt, a “L” represents one litre. For example, the culture vessel may have a volume between 0.3 L and 50 L. For example, the culture vessel may have a volume between 2 L and 10 L. In particular, the culture vessel may preferably have a volume of around 2 L or 5 L. In these examples, the container may be made from plastic such as polypropylene. The 2 L container may provide a similar volume of the enclosed airspace to the 5 L container. Providing culture vessels of at least 2 L allows the bulking up of Sphagnum efficiently, while providing culture vessels of 5 L or less means that the culture vessels are particularly convenient to handle. In some examples, the culture vessel may be a jar e.g. made of glass, which may have a volume of around 300 ml. This is particularly useful at early stages of growth. Larger vessels may be used on a large scale, such as up to 50 L containers, but these are more difficult to use practically. In some cases, flexible sealed bags may be used as the culture vessel. Keeping the culture vessels at smaller volumes also reduces the impact of contamination on the entire culture vessel.

Optionally, the apparatus further comprises a second culture vessel for Sphagnum and a second culture medium arranged in the second culture vessel, wherein the second culture vessel comprises a second enclosed airspace above the second culture medium; and further comprising Sphagnum arranged in the second culture medium; and wherein the means for supplying carbon dioxide is configured to supply carbon dioxide into the second enclosed airspace of the second culture vessel.

The second culture vessel may comprise one or more features described above in relation to the first culture vessel. Equally, the second culture medium may comprise one or more features described above in relation to the first culture medium. By using the same means for supplying carbon dioxide, carbon dioxide can be supplied to both culture vessels from the same source. For example, the apparatus may comprise a second conduit arrangement configured to supply carbon dioxide into the second enclosed airspace. In some examples, the second conduit arrangement may be configured to supply carbon dioxide from the source of carbon dioxide to the second enclosed airspace.

In some examples, the outlet pipe of the first culture vessel is configured to supply carbon dioxide to the second enclosed airspace of the second culture vessel. In this way, the outlet pipe forms a connection between the first enclosed airspace of the first culture vessel and the second enclosed airspace of the second culture vessel. Thus, a series of culture vessels can be connected so that carbon dioxide can be supplied through a conduit connecting each of the culture vessels.

In some examples, the second culture vessel is provided with features of the first culture vessel such as a barrier e.g. in the form of a permeable tube, and optionally an inlet pipe. The second culture vessel can therefore be supplied with carbon dioxide from the first culture vessel, which can then diffuse into the second enclosed airspace in an analogous manner. The second barrier, for example a second tube, can be arranged to permit carbon dioxide into the second enclosed airspace.

For example, the apparatus may comprise a second tube permeable to carbon dioxide, wherein a first end of the second tube is connected to the outlet pipe connected to the first culture vessel. For example, the second tube may be arranged at least partially in the enclosed airspace of the second culture vessel. Therefore, the outlet pipe (or first permeable tube) from the first culture vessel may connect to the second permeable tube of the second culture vessel and supply carbon dioxide into the enclosed airspace of the second culture vessel. The carbon dioxide may diffuse through a wall of the second tube into the enclosed airspace. A plurality of culture vessels can be connected in series in a corresponding manner. This provides a single series flow of carbon dioxide to be supplied to a plurality of connected culture vessels. This allows for more efficient use of the carbon dioxide and a simplified tubing arrangement compared to parallel supply.

In some examples, the second culture medium comprises a second liquid culture medium. This may have features of the liquid culture medium described above.

Optionally, the second culture medium comprises a second liquid culture medium, and the means for stirring is further configured to stir the second liquid culture medium of the second culture vessel. In other words, the same means for stirring is configured to stir the liquid culture medium of the first culture vessel as well as the second culture vessel. This provides a more efficient arrangement as each culture vessel does not require its own dedicated means for stirring. For example, where a heat source is used as the means for stirring, the same heat source can be used to stir the first and second culture vessels, for example arranged between the culture vessels. For example, the same light source may be used.

Optionally, the light source is arranged between the culture vessel and the second culture vessel. This permits the same light source to be used for a plurality of culture vessels. For example, a plurality of culture vessels can be clustered around a light source, such that the heat is applied to a side of each culture vessel. This is particularly useful where the light source is a tube (e.g. a fluorescent tube), as a plurality of light sources can be clustered around the tube (for example, the tube being arranged vertically) to harness the light and heat being emitted in all directions.

Disclosed herein is a system for use in culturing Sphagnum, the system comprising: a first culture vessel for Sphagnum, and a first culture medium arranged in the first culture vessel, wherein the first culture vessel comprises a first enclosed airspace above the first culture medium, and Sphagnum arranged in the first culture medium; a second culture vessel for Sphagnum, and a second culture medium arranged in the second culture vessel, wherein the second culture vessel comprises a second enclosed airspace above the second culture medium, and Sphagnum arranged in the second culture medium; and means for supplying carbon dioxide into the first enclosed airspace of the first culture vessel and the second enclosed airspace of the second culture vessel. This allows a single series flow of carbon dioxide to be supplied to a plurality of culture vessels.

According to a second aspect of the present disclosure, there is provided an apparatus for use in culturing Sphagnum, comprising: a culture vessel for Sphagnum; a liquid culture medium arranged in the culture vessel; Sphagnum arranged in the liquid culture medium; and a light source configured to supply light to the Sphagnum, and further configured to create a temperature differential in the culture vessel to stir the liquid culture medium.

This provides a light source which provides light to the Sphagnum for photosynthesis, improving the growth rate. The light source is also arranged to create a temperature differential in the culture vessel. In other words, the temperature at one point in the culture vessel is higher than at another point. Preferably, the light source is arranged at one side of the culture vessel, and the temperature is higher at the near side of the culture vessel adjacent the light source than at the opposite side furthest from the light source. Preferably, there is not more than one light source (or heat source) arranged adjacent the culture vessel. This improves the temperature differential. A single light source (e.g. a fluorescent tube or an LED tube) can be used for a culture vessel.

By applying a temperature differential, the liquid culture medium is stirred by a convection current from the difference in temperature. Where carbon dioxide is supplied to the culture vessel, this stirring increases diffusion of carbon dioxide into the liquid culture medium, as described above. For example, the temperature differential may be across a width of the culture vessel.

Furthermore, by stirring the liquid culture medium, the uptake of nutrients by the Sphagnum can be increased as the Sphagnum is exposed to different parts of the liquid culture medium and the distribution of nutrients can be made more uniform. Typically, the nutrients close to the Sphagnum will be used up faster, so by stirring the liquid medium the supply of nutrients can be improved.

It is preferable for the lighting arrangement to be asymmetric in that there is a light source adjacent one side of the culture vessel and not adjacent the opposite side in order to provide the temperature differential.

In some examples, the apparatus of the second aspect may comprise one or more features of other aspects. For example, the light source may have features described in relation to other aspects. In one example, the light source is a fluorescent tube. For example, the light source can be arranged to extend across a height of the liquid culture medium. In some examples, the temperature differential is at least 1° C., preferably at least 2° C., more preferably at least 2.5° C., even more preferably at least 3° C.

Preferably, a plurality of culture vessels are arranged to surround the light source such that the light source is configured to supply light to the Sphagnum of the plurality of culture vessels, and further configured to create a temperature differential in each culture vessel to stir the liquid culture medium in each culture vessel. This allows a single light source to be used for a plurality of culture vessels. This is particularly effective where a plurality of culture vessels are arranged to surround a vertically-arranged tube light source, and this also makes good use of space.

Disclosed herein is a system for use in culturing Sphagnum, comprising: a culture vessel for Sphagnum, and a culture medium arranged in the culture vessel, wherein the culture vessel comprises an enclosed airspace above the culture medium, and Sphagnum arranged in the culture medium; and a light source configured to supply light to the culture vessel, wherein the light source is arranged adjacent a first side of the culture vessel, and is arranged to extend across a height of the culture medium.

According to a third aspect of the present disclosure, there is provided a system for use in culturing Sphagnum, comprising: first culture vessel for Sphagnum, and a first liquid culture medium arranged in the first culture vessel, wherein the first culture vessel comprises a first enclosed airspace above the first liquid culture medium, and Sphagnum arranged in the first liquid culture medium; a second culture vessel for Sphagnum, and a second liquid culture medium arranged in the second culture vessel, wherein the second culture vessel comprises a second enclosed airspace above the second liquid culture medium, and Sphagnum arranged in the second liquid culture medium; and a light source configured to supply light to the first culture vessel and the second culture vessel, wherein the light source is arranged between a first side of the first culture vessel and a first side of the second culture vessel, wherein the first side of the first culture vessel and the first side of the second culture vessel are arranged adjacent to each other; and wherein the light source is arranged to extend across a height of the first liquid culture medium and the second liquid culture medium.

In this way, light is supplied to the Sphagnum from the side of each culture vessel. As Sphagnum can be grown in a liquid medium in a culture vessel, light can be provided in directions that are not ordinarily possible, nor desirable, with normal plants. For example, light is conventionally applied from above as the plant is planted in soil or another growing medium. Even in in vitro cultivation of other plants, light is applied from above to become incident on leaves above the growth medium. There is no reason to apply light to the sides of the growth medium, as light is not required or desirable at the roots. However, Sphagnum does not have any roots, and can be cultivated in a liquid medium. Light can be provided from all directions. In particular, light can be provided from the side of the culture vessel. This can improve the supply of light to parts of Sphagnum that are positioned away from the top surface of the culture vessel.

As the light source is arranged to extend across a height of the liquid culture medium, it is ensured that light is supplied over the full height and thus illuminates the Sphagnum.

The height of the culture media refers to the vertical direction, when the culture vessels are placed upright. The light source extending across the height means that the light source has a height at least as tall as the height of the culture media. Preferably, the light source extends across a height of the first culture vessel and the second culture vessel. For example, the light source may be a fluorescent tube which is arranged vertically having a height greater than a height of the culture vessels (e.g. at least 20 cm).

Features described herein in relation to other aspects may be readily applicable to the system of the third aspect. For example, the culture vessels may have similar features to described herein. In some examples, the light source may have similar features described herein.

The light source emits light in a plurality of directions. In some examples, the light source emits light in all directions in the plane perpendicular to the height of the culture vessel. For example, the light source may be generally tubular, with its longitudinal axis arranged parallel to the height of the culture vessel such that light can be emitted radially from the axis. In this manner, light can be provided to a plurality of culture vessels arranged around the light source. In some embodiments, more than two culture vessels can be clustered around the light source to maximise use of the light.

As disclosed herein, in cases where carbon dioxide is supplied to the Sphagnum (such as instead of sugar), the light source can additionally provide a heat source for stirring the liquid medium for uptake of carbon dioxide. In these cases, it may be desirable to only provide a light source at one side of the culture vessel in order to generate a temperature differential for stirring. This is a further advantage to clustering culture vessels around a light source, as the light is only required on a single side.

A preferred arrangement can be provided with a first cluster of culture vessels surrounding a first light source, and a second cluster of culture vessels surrounding a second light source. The first cluster and the second cluster can be arranged adjacent to each other. This means that the second cluster of culture vessels are separated from the first light source by the first cluster of culture vessels and will be separated by a distance at least equal to the width of the culture vessels of the first cluster. This can ensure that the light from the first light source does not provide a significant heating effect on the culture vessels of the second cluster, in order to provide a desired temperature differential caused by the light surrounded by the second cluster. In such arrangements, it is preferable to arrange the light source surrounded by each cluster within a distance from the nearest side of the culture vessels which is less than a width of the culture vessel in order to ensure the culture vessels are closer to the light they are surrounding than to the light of the other cluster. In this way, culture vessels can be arranged efficiently in clusters surrounding a light source, maximising the use of the light, while efficiently arranging a plurality of culture vessels in a small space, which can be important to maximise the use of space in a production facility.

In some examples, the light source is a fluorescent tube. Features and advantages are described herein. Fluorescent tubes are particularly effective as they are energy efficient while providing sufficient light intensity for suitable growth. As they are elongate, they can be arranged to extend across the height of the culture media. The light source can be a fluorescent tube which emits light in all directions about its axis, making efficient use of the light. In other examples, the light source comprises an LED tube.

In some examples, the fluorescent tube is arranged to extend vertically. Optionally, the fluorescent tube is arranged with its axis parallel to the height of the culture vessel.

In some examples, some of the plurality of culture vessels can be arranged at different positions along the axis of the height of the culture vessel. In other words, some of the plurality of culture vessels may be arranged at different vertical positions. For example, some culture vessels may be stacked on others. In other examples, some culture vessels may be arranged on shelves at different heights. In these examples, the light source may be arranged to extend across the culture vessels (stacked, or on different shelves) to extend across a combined height of the culture media (and optionally a combined height of the culture vessels). For example, if the height of a culture vessel is around 20 cm, then when two are stacked, the combined height is around 40 cm, and the light source (e.g. the fluorescent tube) extends a height of at least 40 cm to provide light to both culture vessels. In other words, the same light source can provide light to a plurality of culture vessels at different heights. For example, a first cluster may be arranged surrounding the light source and a second cluster may be arranged surrounding the same light source, wherein the second cluster are stacked on top of the first cluster. In this case, the light source extends across a height of the first cluster and the second cluster. It is particularly useful to use a light tube (e.g. fluorescent tube) where culture vessels can be stacked and clustered around and along the elongate height of the tube.

In one example, multiple culture vessels can be stacked (e.g. at least two, at least four, at least six). However, such stacking may be unstable (especially greater than two) or may restrict access to the culture vessels within the stack. In another example, culture vessels may be arranged on different shelves. On each shelf, the culture vessels may be arranged singly, or in small stacks (e.g. of two or three). For example, each shelf may contain one layer of culture vessels. Preferably, each shelf contains culture vessels stacked two high.

A single light source may be used to supply light to the multiple culture vessels, stacked and/or arranged on shelves. In one example, the system comprises at least two shelves, each having culture vessels stacked two high. A single light source (e.g. a fluorescent tube) may be arranged to extend over the combined height of the system such that light is provided to each of the culture vessels.

As fluorescent tubes can be obtained in sizes of up to 1 m or even 2 m, many culture vessels (stacked or arranged on multiple shelves) can be supplied with light from a single tube arranged vertically.

In some examples, the system is arranged on a trolley, such as a horticulture trolley typically referred to in the art as a Danish trolley. The Danish trolley has a plurality of shelves that can be arranged at the desired height. Moreover, each shelf has two apertures generally for holding. However, the inventors have found that the light source (e.g. a fluorescent tube) can be inserted through aligned apertures of the shelves such that the light source is vertically positioned and held within the trolley. Culture vessels can then be clustered around the vertical light source. Two light sources can therefore be used per trolley. In some cases, shorter light sources can be used over part of the height, and multiple shorter light sources can be attached end to end to provide the required lighting arrangement.

With 5 L culture vessels having a height of around 20 cm, and a width of around 20 cm, it has been found that each shelf can hold 12 culture vessels on a single layer. Each set of 6 culture vessels can surround each light source arranged through the hole in the shelf. It has been found most efficient to provide two layers of culture vessels in a stack per shelf to provide easy access to particular culture vessels. This permits up to four shelves providing a total of 96 culture vessels per trolley.

The Danish trolley has wheels and thus the entire system is movable. This allows the culture vessels to be moved all at once. This provides a more convenient system for a production facility.

The culture vessels may contain any of the features described above, such as the means for supplying carbon dioxide and the pipe arrangement to implement this. It will be understood that features of other aspects can be applied to this aspect in this regard. In some examples, the system comprises tubes permeable to carbon dioxide for each culture vessel. The culture vessels may be connected so that carbon dioxide can be supplied via a single continuous conduit. The light source can act as a heat source to stir and increase diffusion as described herein.

Disclosed herein is a method of culturing Sphagnum, comprising: providing the apparatus as disclosed herein; and supplying carbon dioxide into the enclosed airspace.

According to a fourth aspect of the present disclosure, there is provided a method of culturing Sphagnum, comprising: providing a culture vessel for Sphagnum; providing a culture medium, wherein the culture vessel comprises an enclosed airspace above the culture medium; providing Sphagnum arranged in the culture medium; and supplying carbon dioxide into the enclosed airspace.

It will be understood that features described herein in relation to the apparatus can readily be applied to the method. For example, features of the culture vessel, the culture medium, the Sphagnum, and the supply of carbon dioxide described in relation to the first to third aspects can readily be applied to the method of the fourth aspect.

Optionally, the supplying carbon dioxide comprises supplying a gas comprising at least 90% carbon dioxide by volume. In other examples, the supplying carbon dioxide comprises supplying a gas comprising at least 1% carbon dioxide by volume, such as air with elevated levels of carbon dioxide, optionally at least 50% carbon dioxide by volume.

Optionally, the method further comprises stirring the liquid medium. As above, this can improve uptake of carbon dioxide. For example, the liquid medium may be stirred from externally of the culture vessel. For example, the liquid medium may be stirred by using a heat source. For example, the heat source may comprise a light source. This may comprise one or more features described herein, such as in relation to the first aspect.

Optionally, the liquid medium does not comprise sugar. For example, the method does not comprise culturing the Sphagnum with sugar. In other words, the Sphagnum is cultured in the absence of sugar.

In some examples, the supplying carbon dioxide comprises providing a concentration of carbon dioxide in the enclosed airspace of at least 1,000 ppm, preferably at least 2,500 ppm, more preferably at least 5,000 ppm, even more preferably at least 10,000 ppm, still more preferably at least 20,000 ppm, yet still more preferably at least 30,000 ppm. In some cases, the concentration of carbon dioxide in the enclosed airspace may be up to 50,000 ppm. For example, preferably the concentration of carbon dioxide in the enclosed airspace is between 2,000 ppm and 50,000 ppm, more preferably between 5,000 ppm and 50,000 ppm. The apparatus of other aspects may be configured to supply such concentrations.

In some examples, the method further comprises supplying light to the culture vessel by providing a light source.

In some examples, the method further comprises culturing the Sphagnum in the culture vessel for at least one week, preferably at least four weeks, more preferably at least eight weeks.

Optionally, the method further comprises providing the apparatus as disclosed herein.

Any suitable Sphagnum species (or optionally a combination thereof) may be used in the present disclosure. As different species of Sphagnum may have different growth requirements, the Sphagnum species for use in the present disclosure may be selected depending on the environment.

The Sphagnum may comprise one or more Sphagnum species. Any species could be used, but in one example the present disclosure comprises the use of one or more Sphagnum species selected from the group consisting of: Sphagnum angustifolium, Sphagnum australe, Sphagnum capillifolium, Sphagnum centrale, Sphagnum compactum, Sphagnum cuspidatum, Sphagnum denticulatum, Sphagnum fallax, Sphagnum fimbriatum, Sphagnum fuscum, Sphagnum imbricatum (austinii), Sphagnum inundatum, Sphagnum magellanicum (medium), Sphagnum palustre, Sphagnum papillosum, Sphagnum pulchrum, Sphagnum russowii, Sphagnum squarrosum, Sphagnum subnitens, Sphagnum tenellum, and Sphagnum cristatum. In one example, the method comprises the use of one or more Sphagnum species selected from the group consisting of: Sphagnum palustre, Sphagnum capillifolium, Sphagnum capillifolium rubellum, Sphagnum subnitens, Sphagnum denticulatum, Sphagnum squarrosum, Sphagnum fallax, Sphagnum fimbriatum, Sphagnum cuspidatum, Sphagnum magellanicum, and Sphagnum papillosum. In one example, the invention comprises the use of one or more Sphagnum species selected from the group consisting of: Sphagnum palustre, Sphagnum capillifolium, Sphagnum capillifolium rubellum, Sphagnum subnitens, Sphagnum squarrosum, Sphagnum magellanicum, and Sphagnum papillosum.

In one example, a Sphagnum species for use in the present disclosure may be one or more selected from the group consisting of: Sphagnum palustre, Sphagnum capillifolium, Sphagnum fallax, Sphagnum magellanicum, Sphagnum papillosum, and Sphagnum squarrosum.

Most preferably the Sphagnum species is Sphagnum palustre. For example, Sphagnum palustre may be preferable for use in a growing medium because of its physical properties.

It is also envisaged that the invention could be applied to any hybrid Sphagnum species.

In some examples, the Sphagnum comprises at least one of the Sphagnum species disclosed herein. In some examples, the Sphagnum comprises at least 2, 3, 4, 5 or more Sphagnum species.

Features of one aspect can be readily applied to other aspects. Apparatus features can be readily applied to method features and vice versa. Aspects of the invention may be provided in conjunction with each other and features of one aspect may be applied to other aspects. Any feature in one aspect of the invention may be applied to other aspects of the invention, in any appropriate combination. It should also be appreciated that particular combinations of the various features described and defined in any aspects of the invention can be implemented and/or supplied and/or used independently. Embodiments related to the method may be applied to the Sphagnum obtainable by the method, and vice versa.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Embodiments of the disclosure are described below, by way of example only, with reference to the accompanying Figures.

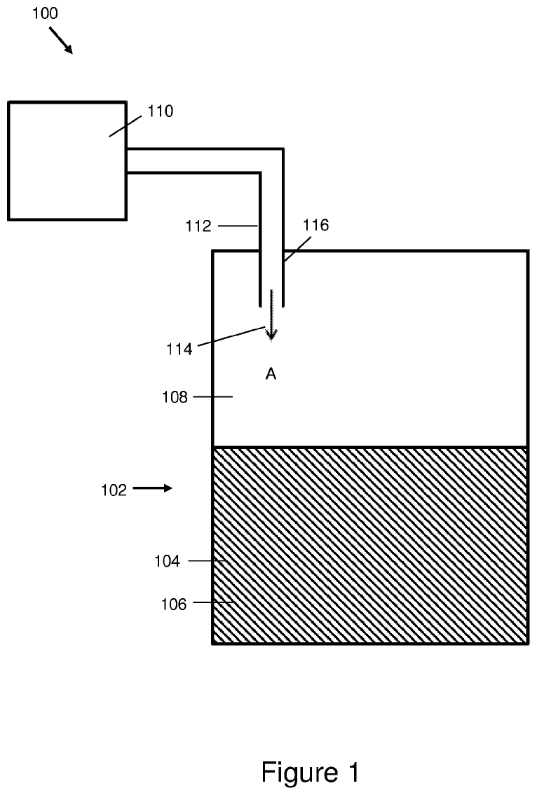

FIG. 1 shows a cross-sectional view of an apparatus for use in culturing Sphagnum, according to a first embodiment of the disclosure.

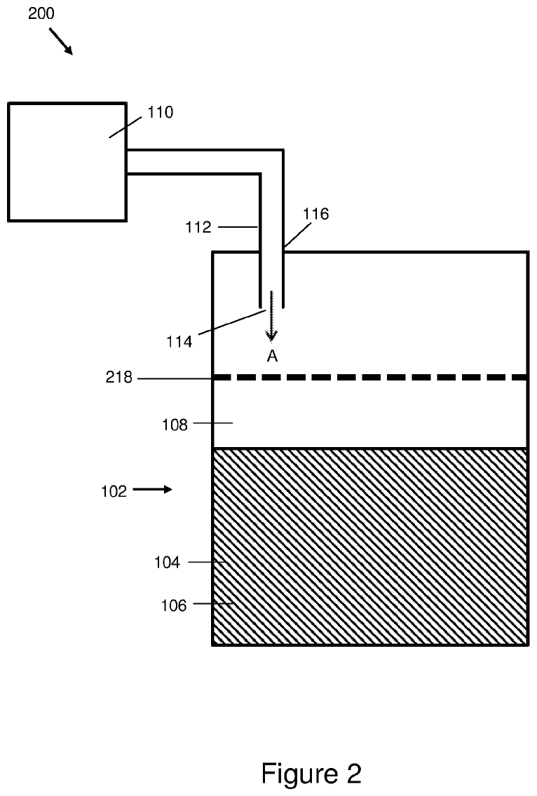

FIG. 2 shows a cross-sectional view of an apparatus for use in culturing Sphagnum, according to a second embodiment of the disclosure, having a permeable membrane.

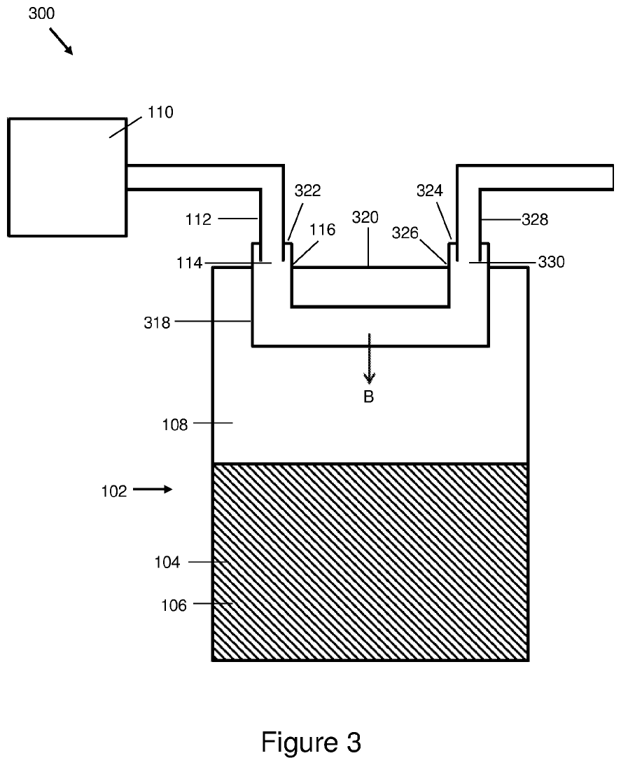

FIG. 3 shows a cross-sectional view of an apparatus for use in culturing Sphagnum, according to a third embodiment of the disclosure, having a permeable tube.

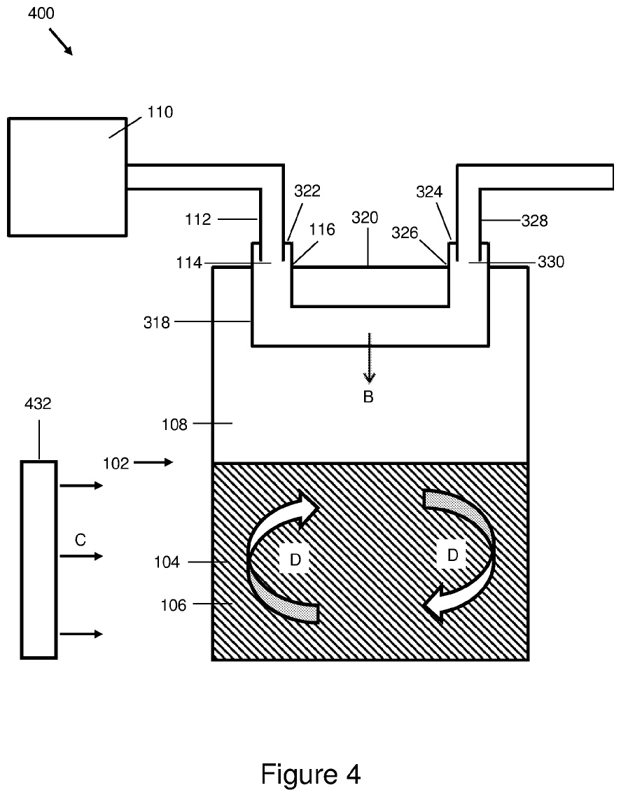

FIG. 4 shows a cross-sectional view of an apparatus for use in culturing Sphagnum, according to a fourth embodiment of the disclosure, having a light source.

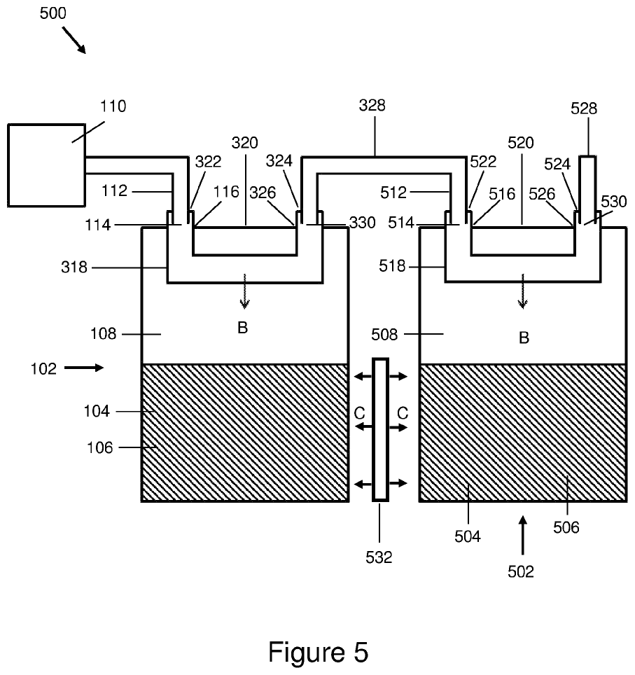

FIG. 5 shows a cross-sectional view of an apparatus for use in culturing Sphagnum, according to a fifth embodiment of the disclosure, comprising a second culture vessel and a shared light source.

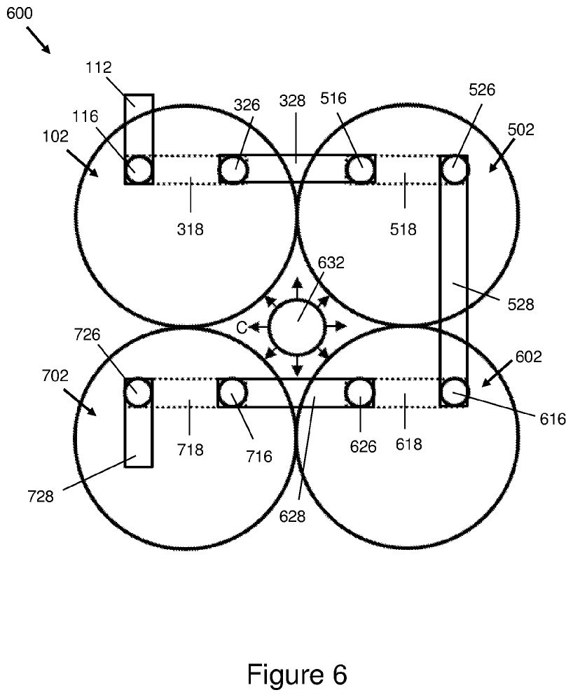

FIG. 6 shows a plan view from above of an apparatus for use in culturing Sphagnum, according to a sixth embodiment of the disclosure, comprising a second, third, and fourth culture vessel and a shared light source.

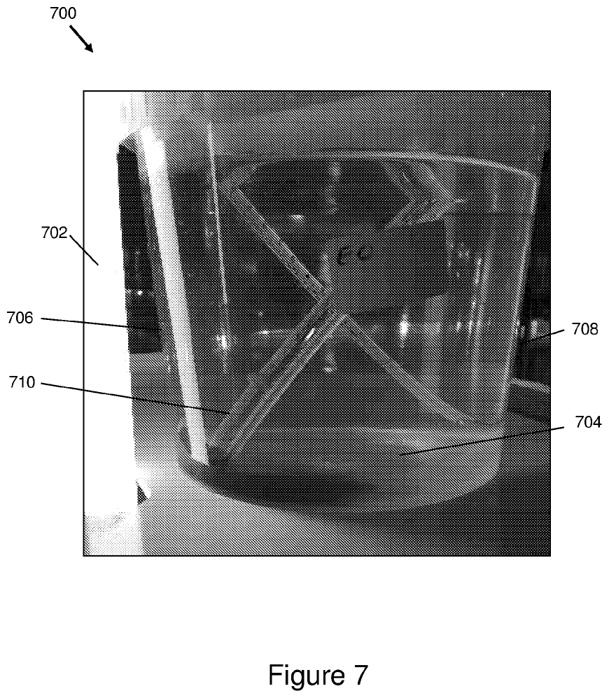

FIG. 7 shows a photograph from the side of an experimental arrangement of a container with water to determine a temperature differential provided by the heating effect of a light source.

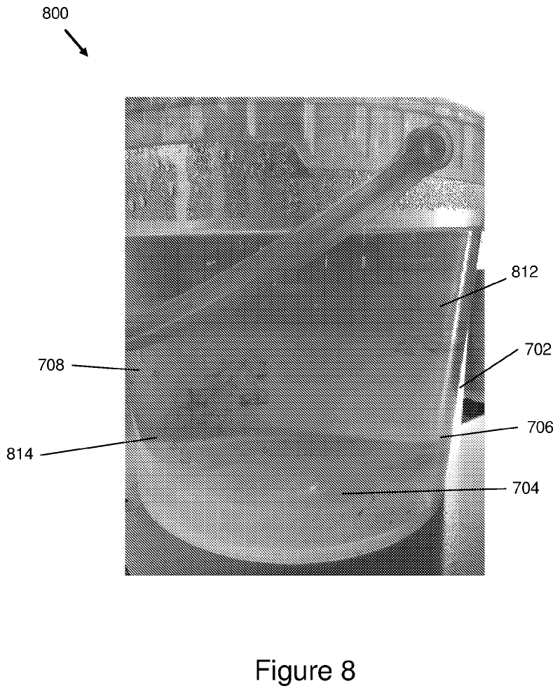

FIG. 8 shows a photograph from the side of an experimental arrangement of a container with water to determine convection currents provided by the heating effect of a light source, with droplets of ink placed into the water.

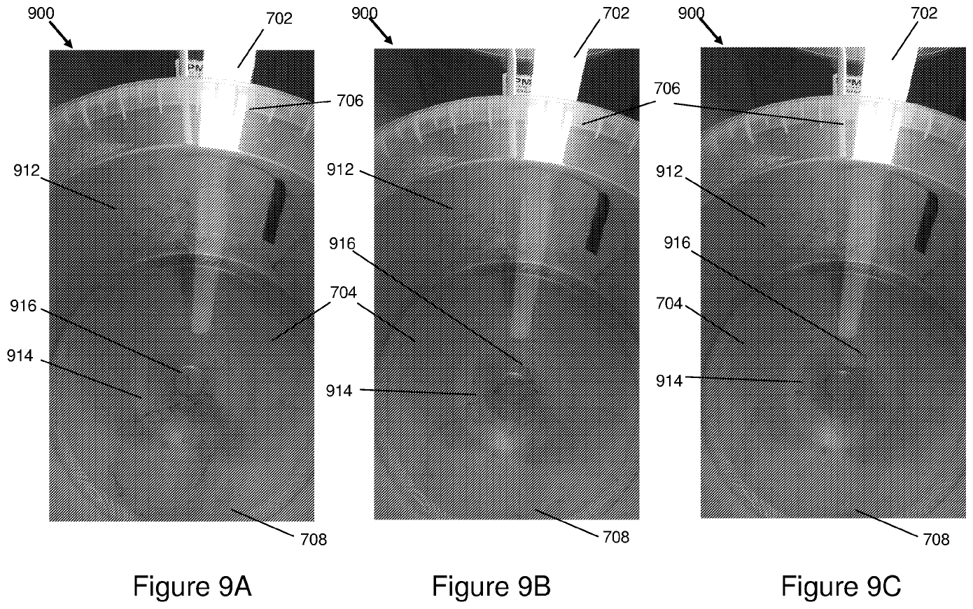

FIG. 9A shows a photograph from above of an experimental arrangement of a container with water to determine convection currents provided by the heating effect of a light source, with droplets of ink placed into the water.

FIG. 9B shows a photograph from above of the experimental arrangement of FIG. 9A, after a predetermined time period.

FIG. 9C shows a photograph from above of the experimental arrangement of FIG. 9B, after a predetermined time period.

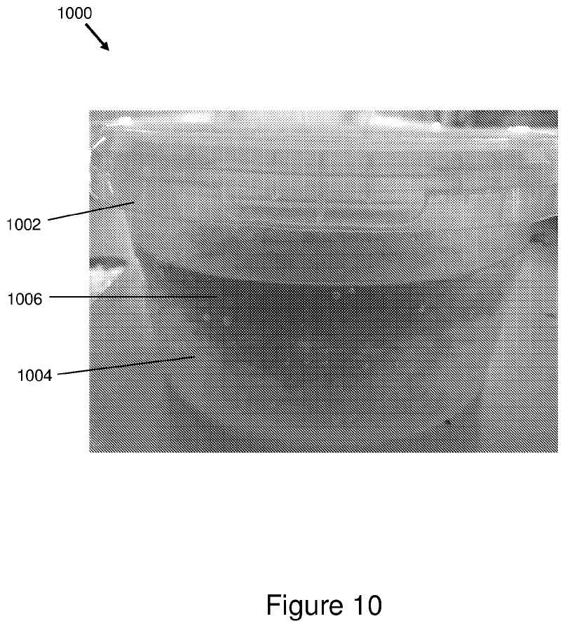

FIG. 10 shows a photograph from the side of an apparatus for use in culturing Sphagnum in accordance with the disclosure.

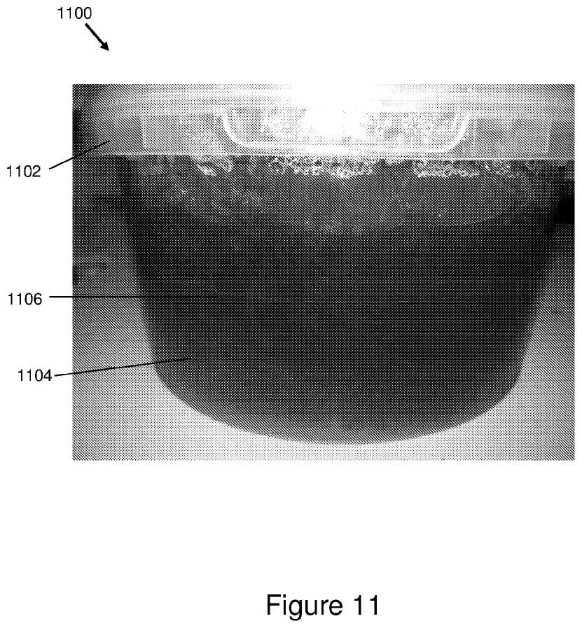

FIG. 11 shows a photograph from the side of the apparatus of FIG. 10, after a period of 12 weeks of growth.

Referring to FIG. 1, according to a first embodiment of the disclosure, an apparatus 100 is provided. The apparatus 100 comprises a culture vessel 102. The culture vessel 102 is a cylindrical container having a circular cross-section, although other shapes of container are envisaged, such as a cube or cuboid. In the first embodiment, the culture vessel 102 is a container made from polypropylene. The culture vessel 102 is transparent, which allows for observation of Sphagnum and visual identification of contamination. The transparent culture vessel 102 also allows for supply of light into the container for photosynthesis. The culture vessel 102 has an interior volume of approximately 5 L, although other volumes are possible such as 2 L or 10 L. The 5 L culture vessel 102 has a height of approximately 195 mm and an upper diameter of about 225 mm.

The culture vessel 102 holds a liquid medium 104 and Sphagnum 106 within the liquid medium 104. In other embodiments, the culture vessel 102 holds a solid medium, for example solidified with agar, with Sphagnum resting on the upper surface of the solid medium. The liquid medium 104 is arranged inside the culture vessel 102, once the culture vessel 102 has been sterilised. In FIG. 1, the liquid medium 104 is shown to occupy approximately half of the volume of the culture vessel 102, although this is primarily to aid understanding of the disclosure. In other examples, the liquid medium 104 can occupy other proportions of the volume of the culture vessel 102, such as around 80% of the volume.

The liquid medium 104 is an aqueous solution comprising water. The liquid medium 104 also comprises nutrients for facilitating the cultivation of Sphagnum. In the first embodiment, the nutrients comprise nitrogen, phosphorus, potassium, calcium, magnesium, sodium, manganese, copper, zinc, sulfur, boron, iron, molybdenum, chlorine, cobalt, and iodine. Different levels of nutrients may be provided in other examples. In alternative examples, some nutrients may be omitted or other nutrients may be included. In the first embodiment, the liquid medium 104 does not comprise sugar (e.g. sucrose). Thus, the Sphagnum 106 is grown in the absence of sugar.

The culture vessel 102 comprises an airspace 108 above the liquid medium 104. The airspace 108 may otherwise be referred to as a headspace. The airspace 108 is a region of the interior volume of the culture vessel 102 in which the liquid medium 104 is not present. For example, if the liquid medium 104 occupies 80% of the volume, then the airspace occupies the remaining space, i.e. 20% of the volume. The airspace 108 is arranged above the liquid medium 104.

In the first embodiment, the Sphagnum 106 is in vitro Sphagnum. The Sphagnum 106 is in the form of strands of whole plants which have been micropropagated. This provides clean material which reduces contamination.

The apparatus 100 comprises a source of carbon dioxide 110. In the first embodiment, the source of carbon dioxide 110 is a bottle of compressed carbon dioxide. In the first embodiment, the source of carbon dioxide 110 is a cylinder of compressed carbon dioxide in liquid form, commercially available from BOC, UK. The source of carbon dioxide 110 is arranged to supply substantially pure (at least 99%) carbon dioxide. In some embodiments, the source of carbon dioxide 110 is arranged to supply a gas comprising at least 1% carbon dioxide by volume, preferably at least 2%, more preferably at least 3%, even more preferably at least 5%, still more preferably at least 50%, yet still more preferably at least 75%, and still further more preferably at least 90%.

The apparatus 100 also comprises an inlet pipe 112. The inlet pipe 112 is connected to the source of carbon dioxide 110. The inlet pipe 112 has an open end 114. The open end 114 is the opposite end of the inlet pipe 112 to the end connected to the source of carbon dioxide 110. The open end 114 is arranged to extend into the culture vessel 102. In other embodiments, the inlet pipe 112 comprises a plurality of serially connected pipes from the source of carbon dioxide 110 to the open end 114 arranged in the culture vessel 102.

The inlet pipe 112 is inserted into the culture vessel 102. In particular, the inlet pipe 112 is inserted through an inlet hole 116 in the wall of the culture vessel 102. In FIG. 1, the inlet hole 116 is in the upper surface of the culture vessel 102, but in other examples it may be in the side wall of the culture vessel 102. In other embodiments, the culture vessel 102 comprises a removable lid, in which case the inlet hole 116 may be in the removable lid. The inlet pipe 112 is inserted through the inlet hole 116 of the culture vessel 102 such that the open end 114 is arranged inside the culture vessel 102, and in particular in the airspace 108. The inlet hole 116 is substantially gas-tight sealed around the inlet pipe 112. Thus, the culture vessel 102 is isolated from the external environment and is sealed except through the connection to the source of carbon dioxide 110 through the inlet pipe 112. In this way, the airspace 108 is enclosed.

The source of carbon dioxide 110 is therefore in fluid communication with the airspace 108 through the inlet pipe 112. Carbon dioxide can be supplied from the source of carbon dioxide 110 to the airspace 108 of the culture vessel 102 via the inlet pipe 112. Arrow A in FIG. 1 indicates the flow of carbon dioxide from the inlet pipe 112 through the open end 114 and into the airspace 108.

The inlet pipe 112 is shown as an L-shape in FIG. 1 for illustrative purposes only, and the inlet pipe 112 may take any shape, and in the first embodiment is a flexible pipe. The inlet pipe 112 is impermeable to carbon dioxide. Thus, carbon dioxide entering the inlet pipe 112 at the source of carbon dioxide 110 will not leak through the walls of the inlet pipe 112, and will only exit through the open end 114. In the first embodiment, the inlet pipe 112 is made from nylon.

Although not illustrated in FIG. 1, in some examples, the inlet pipe 112 may comprise one or more valves for controlling the flow of carbon dioxide from the source of carbon dioxide 110.

In this arrangement, the apparatus 100 constitutes an apparatus for use in culturing the Sphagnum 106. The inlet pipe 112 provides the supply of carbon dioxide into the airspace 108. This allows the concentration of carbon dioxide in the airspace 108 to increase over time. The carbon dioxide in the airspace can be absorbed into the liquid medium 104 by diffusion. The rate of absorption is determined by the surface area of the liquid medium 104 in contact with the airspace 108. Due to the supply of carbon dioxide, and the lack of sugar in the liquid medium 104, the Sphagnum is provided with a carbon source for photosynthesis and risk of contamination is mitigated in accordance with the present disclosure.

In alternative embodiments, the apparatus 100 may further comprise an outlet pipe for removing gas from the airspace 108. This allows for the balancing of pressure within the airspace 108, and allows the airspace 108 to be replenished with carbon dioxide.

Referring to FIG. 2, according to a second embodiment of the disclosure, an apparatus 200 is provided. The apparatus 200 of the second embodiment may include one or more features described above in relation to the first embodiment. The same reference numerals are used to denote identical features.

The apparatus 200 of the second embodiment is similar to the apparatus 100 of the first embodiment shown in FIG. 1, except that the apparatus 200 further comprises a membrane 218. The membrane 218 is permeable to carbon dioxide. The membrane 218 is a barrier permeable to carbon dioxide. In the second embodiment, the membrane 218 is made from silicone rubber, although other materials may be used in other examples. The membrane 218 is arranged inside the culture vessel 102. In the second embodiment, the membrane 218 extends over the entire width of the culture vessel 102 such that the membrane 218 divides the volume of the culture vessel 102 into two regions (an upper region and a lower region). The membrane 218 extends perpendicularly to the height of the culture vessel 102, such that the membrane 218 is arranged substantially horizontally across the width of the culture vessel 102. The lower region contains the liquid medium 104 and the Sphagnum 106 therein. Therefore, the membrane 218 is arranged above the liquid medium 104. The membrane 218 is arranged in contact with the enclosed airspace which is defined between the liquid medium 104 and the membrane 218.

The inlet pipe 112 is inserted through the upper surface of the culture vessel 102 into the upper region above the membrane 218. Thus, the inlet pipe 112 supplies carbon dioxide into a region of the culture vessel 102 above the membrane 218.

The membrane 218 acts as a barrier to separate the liquid medium 104 from the source of carbon dioxide 110. The membrane 218 acts to filter the supplied carbon dioxide to ensure that contaminants are blocked from coming into contact with the liquid medium 104. Carbon dioxide can then diffuse across the membrane 218, while contaminants cannot pass across the membrane 218. This acts as a simple and convenient method of supplying carbon dioxide to the airspace 108, while providing additional protection against contamination. This can be more cost effective than supplying sterilised carbon dioxide.

In alternative embodiments, the membrane 218 may be arranged over the open end 114 of the inlet tube 112. For example, the membrane 218 may be in the form of a cap over the open end 114 of the inlet tube 112. This would then force the carbon dioxide to diffuse through the membrane 218 at the end of the inlet tube 112, isolating the airspace 108 from the source of carbon dioxide 110.

In other embodiments, the membrane 218 may be arranged as the lid of the culture vessel 102. Carbon dioxide can then be supplied through the membrane 218, such as by elevating carbon dioxide levels surrounding the culture vessel 102 (e.g. in the room, or in a container containing one or more culture vessels 102).

Referring to FIG. 3, according to a third embodiment of the disclosure, an apparatus 300 is provided. The apparatus 300 of the third embodiment may include one or more features described above in relation to the first or second embodiments. The same reference numerals are used to denote identical features.

The apparatus 300 of the third embodiment is similar to the apparatus 200 of the second embodiment shown in FIG. 2, except that the apparatus 300 comprises a tube 318 instead of the membrane 218. The tube 318 is a specific embodiment of a permeable barrier.

The apparatus 300 of the third embodiment differs from the first and second embodiments in that the open end 114 of the inlet pipe 112 does not open into the culture vessel 102. Instead, the tube 318 is arranged at least partially within the airspace 108 of the culture vessel 102. The tube 318 passes through the inlet hole 116 in the culture vessel 102. In the third embodiment, the culture vessel 102 has a removable lid 320. In some embodiments, the removable lid 320 need not be provided, and the inlet hole 116 can be arranged in the upper surface of the culture vessel 102 as in the first and second embodiments. The inlet hole 116 is arranged through the removable lid 320, but is otherwise similar to the inlet hole 116 of the first and second embodiments, and a gas-tight seal is formed around the tube 318 at the inlet hole 116. The removable lid 320 can readily be applied to the first and second embodiments.

The tube 318 is connected to the inlet pipe 112. In particular, an inlet end 322 of the tube 318 is connected to the open end 114 of the inlet pipe 112. In the third embodiment, the tube 318 fits over the inlet pipe 112 such that a substantially gas-tight seal is formed. The tube 318 is made from a flexible and deformable material such that it can form a friction fit over the inlet pipe 112 to form a seal.

In the third embodiment, the connection between the inlet pipe 112 and the tube 318 is arranged outside of the culture vessel 102. This facilitates easy connection and disconnection of the inlet pipe 112 and the tube 318. Furthermore, because the interior of the culture vessel 102 is often under sterile conditions, avoiding exposing the Sphagnum 106 to the external environment is desirable, so having the connection accessible without removing the lid 320 is desirable.

In the third embodiment, the tube 318 also has an outlet end 324 at the opposite end of the tube 318 to the inlet end 322. The tube 318 thus extends between the inlet end 322 and the outlet end 324. The outlet end 324 is arranged to extend through an outlet hole 326 in the culture vessel 102. The outlet hole 326 is also arranged in the removable lid 320 of the culture vessel 102. The outlet hole 326 may be similar to the inlet hole 116, and for example is gas-tight sealed around the tube 318.

The apparatus 300 also comprises an outlet pipe 328. The outlet pipe 328 is similar to the inlet tube 112 and is also impermeable to carbon dioxide. In the third embodiment, the outlet pipe 328 is made from nylon. The outlet pipe 328 has an open end 330 which is connected to the outlet end 324 of the tube 318. The tube 318 therefore forms a conduit between the inlet pipe 112 and the outlet pipe 328. In this way, the tube 318 provides a duct from the inlet hole 116 to the outlet hole 326 through the airspace 108 of the culture vessel 102. The duct is a closed loop section, with either end of the tube 318 passing through the removable lid 320.

The tube 318 is permeable to carbon dioxide. In the third embodiment, the tube 318 is made from silicone rubber. Carbon dioxide supplied from the source of carbon dioxide 110 can flow through the inlet pipe 112 and into the tube 318. The carbon dioxide can then diffuse through the wall of the silicone tube 318 and into the airspace 108. This diffusion is indicated by Arrow B in FIG. 3.

In the third embodiment, the tube 318 has a length of around 15 cm, with a length of the part of the tube 318 arranged in the airspace of around 10 cm. In the third embodiment, the inner diameter of the tube is 3 mm. In the third embodiment, the thickness of the tube is 1 mm.

As the tube 318 is also connected to the outlet pipe 328, carbon dioxide that does not diffuse through the permeable tube 318 will be output into the outlet pipe 328. After leaving the tube 318, the outlet pipe 328 can then channel the carbon dioxide where desired. For example, the outlet pipe 328 may be connected to a liquid reservoir where the carbon dioxide is bubbled out of the outlet pipe 328 and into the liquid. In other examples, as described below in relation to the fifth embodiment, the outlet pipe 328 may be connected to another culture vessel 502 to provide a continuous and serial supply of carbon dioxide to multiple culture vessels 102, 502 in a row.

In alternative embodiments, the apparatus 300 does not comprise an outlet pipe 328 and an outlet hole 326. Instead, the tube 318 is sealed at one end such that carbon dioxide is forced to diffuse through the permeable tube 318 into the airspace 108.

Referring to FIG. 4, according to a fourth embodiment of the disclosure, an apparatus 400 is provided. The apparatus 400 of the fourth embodiment may include one or more features described above in relation to any of the first to third embodiments. The same reference numerals are used to denote identical features.

In particular, the apparatus 400 of the fourth embodiment is similar to the apparatus 300 of the third embodiment shown in FIG. 3, except that the apparatus 400 further comprises a light source 432.

In the fourth embodiment, the light source 432 is a white fluorescent tube having a power of 36 W. In other embodiments, the light source 432 may comprise one or more light emitting diodes (LEDs).

The light source 432 is arranged at one side of the culture vessel 102. In particular, the light source 432 is only arranged at one side of the culture vessel 102, and there is no light source arranged on the opposite side. The light source 432 is arranged at a distance less than a width of the culture vessel 102 away from the side of the culture vessel 102.

The light source 432 provides light to the Sphagnum for growth by photosynthesis. The general direction of light emitted towards the culture vessel 102 is indicated by Arrows C. Although not shown, light is emitted in all directions around the longitudinal axis of the fluorescent tube of the light source 432.

The light source 432 is arranged to extend over a height equal to or greater than the height that the liquid medium 104 extends in the culture vessel 102. Thus, the light source 432 is arranged to supply light to Sphagnum 106 throughout the entire height of the liquid medium 104. In alternative embodiments, the light source 432 is arranged to extend over a height equal to or greater than the height of the culture vessel 102.

Efficient use of the light source 432 can be made by arranging a cluster of culture vessels 102 around the light source 432 as will be described below with respect to the sixth embodiment.

In the fourth embodiment, the light source 432 also acts as a heat source. Light source 432 inherently has inefficiencies in converting electrical power into light, resulting in waste heat. The light source 432 thereby heats up the near side of the culture vessel 102. Because the light source 432 is arranged towards one side, it heats up the near side of the culture vessel 102 more than the far side. This heating effect is sufficient to create a temperature differential across the culture vessel 102.

The temperature differential is significant enough to create convection currents in the liquid medium 104. Liquid towards the near side is heated more, and therefore rises towards the surface. Liquid at the top is displaced by more rising liquid, and is pushed away from the near side towards the far side. As it moves over the top surface away from the heat source, it cools and sinks back down to the bottom, and is further displaced by heated liquid. This is then, in turn, displaced by cooling liquid, and is pushed away from the far side towards the near side along the bottom surface. Thus, a convention current is generated generally as indicated by Arrows D.

The convection currents cause a stirring effect with the liquid medium 104 and increase the uptake of carbon dioxide from the airspace 108 into the liquid medium 104. By agitating fresh liquid medium 104 into contact with the airspace 108, saturation can be avoided and the efficiency of the uptake of carbon dioxide by the liquid medium 104 can be increased. By stirring the liquid medium 104 in this way, the diffusion gradient is kept high and the rate of diffusion can be improved. This means that the rate of supply of carbon dioxide to the liquid medium 104 containing the Sphagnum 106 can be increased.

Referring to FIG. 5, according to a fifth embodiment of the disclosure, an apparatus 500 is provided. The apparatus 500 of the fifth embodiment may include one or more features described above in relation to any of the first to fourth embodiments. The same reference numerals are used to denote identical features.

The apparatus 500 of the fifth embodiment comprises all of the features of the apparatus 400 of the fourth embodiment. In overview, the apparatus 500 comprises the apparatus 400 connected to a second apparatus having similar features.

In particular, the apparatus 500 comprises a second culture vessel 502 which is similar to the first culture vessel 102. The second culture vessel 502 contains a liquid medium 504 and Sphagnum 506 therein. The second culture vessel 502 contains an airspace 508 above the liquid medium 504.

The apparatus 500 also contains an inlet pipe 512 which has an open end 514. In the fifth embodiment, the inlet pipe 112 is the distal end of the outlet pipe 328 from the first culture vessel 102. In alternative embodiments, the inlet pipe 512 is a separate pipe which is connected to the outlet pipe 328.

In a corresponding manner to the first culture vessel 102, the second culture vessel 502 also comprises an inlet hole 516 and an outlet hole 526 in a lid 520. A tube 518 extends between the inlet hole 516 and the outlet hole 526 by attaching between the open end 514 of the inlet pipe 512 and the open end 530 of an outlet pipe 528. The tube 518 is permeable to carbon dioxide in the same way as the tube 318.

The first culture vessel 102 is therefore connected to the second culture vessel 502 by virtue of the outlet pipe 328 of the first culture vessel 102 connecting to the tube 518 of the second culture vessel 502. Therefore, carbon dioxide is supplied from the source of carbon dioxide 110 to the first culture vessel 102 and then successively to the second culture vessel 502. Carbon dioxide which does not diffuse out of the tube 318 into the airspace 108 in the first culture vessel 102 can then be supplied for diffusion into the second culture vessel 102 through the tube 518 into the airspace 508.

The apparatus 500 comprises a shared light source 532 arranged between the first culture vessel 102 and the second culture vessel 502. As the light source 532 is arranged closer to one side of each of the first culture vessel 102 and the second culture vessel 502, a convection current can be generated in each of the first culture vessel 102 and the second culture vessel 502, as described above.

In this way, carbon dioxide can be supplied to a plurality of culture vessels 102, 502 by connecting the culture vessels 102, 502 in sequence. Carbon dioxide is thus supplied in a continuous conduit via the inlet pipe 112 of the first culture vessel 102, the tube 318 of the first culture vessel 102, the outlet pipe 328 of the first culture vessel 102, the inlet pipe 512 of the second culture vessel 502, and the tube 518 of the second culture vessel 502. Therefore, because of the diffusion through the walls of the tubes 318, 518, carbon dioxide can be supplied by connections of pipes in series, rather than having a dedicated source of carbon dioxide 110 for each culture vessel 102, 502. This allows for easy scaling up of the number of culture vessels 102, 502, and for a modular design whereby more or fewer culture vessels 102, 502 can be added to the sequence.

In alternative embodiments, the outlet pipe 528 of the second culture vessel 502 is not closed at its end. Instead, the outlet pipe 528 is connected to an inlet pipe of a third culture vessel, to link to a further culture vessel and supply carbon dioxide in a corresponding manner.

Referring to FIG. 6, according to a sixth embodiment of the disclosure, an apparatus 600 is provided. The apparatus 600 of the sixth embodiment may include one or more features described above in relation to any of the first to fifth embodiments. The same reference numerals are used to denote identical features.

The apparatus 600 of the sixth embodiment comprises all of the features of the apparatus 500 of the fifth embodiment. In overview, the apparatus 600 comprises the apparatus 500 connected to a third apparatus and a fourth apparatus having similar features.

FIG. 6 shows the apparatus in a plan view from above. In particular, the apparatus 600 of the sixth embodiment comprises a first culture vessel 102 and a second culture vessel 502 similar to the fifth embodiment. The first culture vessel 102 is connected to the second culture vessel 502 in a similar manner to the fifth embodiment. In particular, the first culture vessel 102 has an inlet hole 116 and an outlet hole 326 in the upper surface. The first culture vessel 102 has a permeable tube 318 arranged between the inlet 116 and the outlet 326 and extending within the enclosed airspace of the culture vessel 102. The tube 318 is illustrated in phantom in FIG. 6 to demonstrate it is below the upper surface (e.g. the lid) of the culture vessel 102. The tube 318 is connected to an inlet pipe 112 at the inlet 116. The tube 318 is also connected to an outlet pipe 328 at the outlet 326. The outlet pipe 328 forms an inlet pipe to the second culture vessel 502. The outlet pipe 328 is connected to a permeable tube 518 at an inlet 516 of the second culture vessel 502. The tube 518 extends from the inlet 516 to an outlet 526, where it is connected to an outlet pipe 528.

The apparatus 600 also comprises a third culture vessel 602 and a fourth culture vessel 702. Each of the third culture vessel 102 and fourth culture vessel 102 is similar to the culture vessels 102, 502 of the fifth embodiment.

The second culture vessel 502 is connected to the third culture vessel 602 in a similar manner to the connection of the first culture vessel 102 to the second culture vessel 502. In particular, the outlet pipe 528 is connected to a permeable tube 618 of the third culture vessel 602 at an inlet 616. The tube 618 extends between the inlet 616 and an outlet 626. The tube 618 is connected to an outlet pipe 628 at the outlet 626.

The third culture vessel 602 is connected to the fourth culture vessel 702 in a similar manner to the connection of the first culture vessel 102 to the second culture vessel 502 and the connection of the second culture vessel 502 to the third culture vessel 602. In particular, the outlet pipe 628 is connected to a permeable tube 718 of the fourth culture vessel 702 at an inlet 716. The tube 718 extends between the inlet 716 and an outlet 726. The tube 718 is connected to an outlet pipe 728 at the outlet 726.

In the sixth embodiment, the culture vessels 102, 502, 602, 702 are arranged in contact with adjacent culture vessels 102, 502, 602, 702. In particular, the culture vessels 102, 502, 602, 702 are arranged in a loop or cluster so that the fourth culture vessel 702 is in contact with the third culture vessel 602 and the first culture vessel 102. In other embodiments, the culture vessels 102, 502, 602, 702 need not be in contact.

The apparatus 600 also includes a light source 632 arranged between the first culture vessel 102, the second culture vessel 502, the third culture vessel 602, and the fourth culture vessel 702. In other words, the culture vessels 102, 502, 602, 702 are clustered around the light source 632. The light source 632 is a fluorescent tube similar to the third to fifth embodiments, and is arranged vertically generally parallel to a height of the culture vessels 102, 502, 602, 702.

In this manner, space can be utilised whilst achieving the advantages described herein. Light is emitted out of the light source 632 in 360° as indicated by Arrows C. The plurality of culture vessels 102, 502, 602, 702 are arranged to surround the light source 632 to maximise use of the light.

In alternative embodiments, other numbers of culture vessels can be arranged around the light source 632. For example, more than four culture vessels 102, 502, 602, 702 can surround a single light source 632. In one example, six culture vessels can surround a light source 632.

In alternative embodiments, another cluster of culture vessels may additionally be provided. This cluster can be arranged around another light source. Because the culture vessels 102, 502, 602, 702 of the first cluster are separated from the light source surrounded by the second cluster, the light source of the second cluster has far less effect than the light source 632 of the first cluster. This ensures a temperature gradient is provided as desired. The first cluster may be connected to the second cluster to provide a supply of carbon dioxide.

In alternative embodiments, further culture vessels can be stacked on top of each other (e.g. providing eight culture vessels by stacking two layers of the cluster of four culture vessels). This can provide better use of space, especially where a fluorescent tube is used as the light source 632 and the tube is at least the height of two culture vessels. This provides light into the height of the liquid medium 104 in each culture vessel.

The arrangement of the sixth embodiment not only provides an optimum use of space and ensures an even use of light from the vertical tubes, but it also efficiently supplies carbon dioxide to a plurality of culture vessels 102, and also provides a heat source at one side of each culture vessel 102 in an effective manner to stir the liquid media 104.

In one example, 96 culture vessels each of 5 L volume are arranged on a Danish trolley, with 6 culture vessels clustered around each tube light source (with two light sources arranged through the two holes in the trolley shelves) to provide 12 culture vessels on each shelf, with each shelf having culture vessels stacked two high, and providing four shelves per trolley. Therefore 96 culture vessels can be cultured using two light tubes, and supply of carbon dioxide can be supplied by serially connected each culture vessel.

### EXAMPLES

### Example 1

Materials and Methods

A trial was set up to determine the effects of providing a light source as a heat source in order to generate a convection current for stirring a liquid culture medium for culturing Sphagnum. FIG. 7 shows the arrangement 700 of the experimental setup.

A light source 702 was provided in the form of a fluorescent tube. The fluorescent tube was a 36 W tube emitting white light. The light source 702 can be seen towards the left-hand side of FIG. 7.

A culture vessel in the form of a 5 L polypropylene container 704 was partially filled with water to represent a liquid culture medium. Sphagnum was omitted from the water to aid visual indication of currents. The container 704 was positioned adjacent to the light source 702. The rim of the lid of the container 704 was placed as close to the light source 702 as possible without touching. The container 704 was arranged so that the light source 702 was arranged at the near side 706, but there was no other light or heat source arranged adjacent the opposite far side 708 of the container 704.

The temperature was measured using a mercury thermometer 710 located at the bottom corner of the water at the near side 706 nearest the light source 702 and at the far side 708 furthest from the light source 702. A thermometer 710 was positioned at each location to provide constant readings to detect any variations.

Liquid ink was then administered to the water in the container 704 to act as a visual indicator to demonstrate any convection current. A narrow plastic tube was used to hold liquid ink via capillary action. The tube was then lowered into contact with the upper surface of the water, the suction force drawing the ink out in a consistent manner. Two drops were placed onto the surface at the same time: one towards the near side 706 nearest the light source 702, and one towards the far side 708 furthest from the light source 702.

Once the drops were administered, the motion of the drops was observed to indicate currents within the water.

Results

For the container 704 adjacent the light source 702, the temperature at the far side 708 (furthest from the light source 702) was measured to be 20° C. The temperature at the near side 706 (nearest the light source 702) was measured to be 23° C., and fluctuated between 22.5° C. and 23° C. throughout the experiment. This provided a temperature differential across the width of the container 704 of 2.5° C. to 3° C.

The drop in the container 704 at the near side 706 adjacent the light source 702 was observed to behave differently to the drop at the far side 708. In particular, the droplet at the near side 706 was slower to sink to the bottom than the droplet at the far side 708.

FIG. 8 shows a photograph of the arrangement 800 similar to the arrangement 700 of FIG. 7 after administering droplets of ink onto the water, the droplets of ink 812 and 814 visible in contrast to the colourless water. The light source 702 can be seen towards the right-hand side of FIG. 8. After a predetermined time period, the droplet of ink on the near side 706 had sunk less than at the far side 708. FIG. 8 shows a snapshot after the predetermined time period, and shows the droplet 812 at the near side 706 towards the light source 702 towards the right-hand side of FIG. 8, and the droplet 814 at the far side 708 towards the left-hand side of FIG. 8. As shown, the droplet 812 at the near side 706 was observed to sink less than the droplet 814 at the far side 708. This confirmed that a convection current was being generated within the water to cause an upward movement on the near side 706 due to heating by the light source 702 and a downward movement on the far side 708. Due to the heat from the light source 702, the near side 706 experiences a buoyancy effect due to the rising warmer liquid.

The droplet 814 at the far side 708 was then observed to diffuse along the bottom towards the near side 706, whereas the droplet 812 at the near side 706 began to diffuse upwards and eventually along the upper surface of the water towards the far side 708. The movement of the droplet 812 was observed to be slower than the droplet 814 because the convention current had to overcome the weight of the ink, which was heavier than the water.

FIGS. 9A to 9C show successive photographs in time from above of the arrangement 900 similar to the arrangement 800 of FIG. 8. The light source 702 is shown towards the top of FIGS. 9A to 9C, where the near side 706 of the container 704 is shown towards the top and the far side 708 is shown towards the bottom.

FIG. 9A shows a snapshot of the droplet 912 at the near side 706 and the droplet 914 at the far side 708 after a predetermined time period after dropping the droplets 912, 914 into the water. The droplet 912 can be seen at the upper surface of the water in the container 704, whereas the droplet 914 can be seen at the bottom surface of the container 704. The droplets 912, 914 are beginning to diffuse into a cloud of ink. The droplet 914 at the far side 708 can be seen to be present at a furthest point 916 towards the near side 706 around the position of the centre circle of the container 704.

FIG. 9B shows a snapshot a predetermined time period after the snapshot of FIG. 9A. The droplet 914 at the far side 708 can be seen to have moved relative to the snapshot of FIG. 9A. In particular, the droplet 914 has moved towards the near side 706, and can be seen to be present at a furthest point 916 which is beyond the centre circle of the container 704. The ink droplet 914 has moved in the direction from the far side 708 towards the near side 706 along the bottom surface of the container 704. The droplet 912 is also shown to have moved away slightly from the near side 706 towards the far side 708.

FIG. 9C shows a snapshot a predetermined time period after the snapshot of FIG. 9B. The droplet 914 at the far side 708 can be seen to have moved relative to the snapshot of FIG. 9B. In particular, the droplet 914 has moved towards the near side 706, and can be seen to be present at a furthest point 916 which is well beyond the centre circle of the container 704. The ink droplet 914 has moved in the direction from the far side 708 towards the near side 706 along the bottom surface of the container 704. The droplet 912 is also shown to have moved further away from the near side 706 towards the far side 708.

The droplet 914 diffused along the bottom surface of the container 704. As it reached the near side 702, it began to rise towards the upper surface. This clearly demonstrates the convention current which stirs the water due to the temperature differential applied by the light source 702 at the near side 706.

### Example 2

Material and Methods

A trial was conducted using an apparatus similar to that described above in relation to FIG. 4. Sphagnum was placed into a liquid culture medium within a 2 L culture vessel. The liquid culture medium comprised nutrients comprising 48.23 mg per L of nitrogen, 9.67 mg per L of phosphorus, 123.46 mg per L of potassium, 32.63 mg per L of calcium, 9.12 mg per L of magnesium, 1.32 mg per L of sodium, 5.49 mg per L of manganese, 0.02 mg per L of copper, 1.96 mg per L of zinc, 62.55 mg per L of sulfur, 1.08 mg per L of boron, 1.40 mg per L of iron, mg per L of molybdenum, 11.69 mg per L of chlorine, 0.006 mg per L of cobalt, and 0.63 mg per L of iodine. There was no sugar in the culture medium.

A permeable tube made from silicone rubber was inserted through a lid of the culture vessel. The permeable tube had a length of around 15 cm, with 10 cm within the interior of the culture vessel. The tube had an inner diameter of 3 mm and a wall thickness of 1 mm. The tube was formed into a loop, threaded into and out through the lid of the culture vessel. One end of the tube was connected to a nylon inlet pipe which was in turn connected to a source of carbon dioxide in the form of a canister of carbon dioxide (at least 99% pure), commercially available from BOC, UK. The other end of the tube was connected to a nylon outlet pipe, with the distal end of the outlet pipe placed into a jar of water to bubble through as an outlet. The carbon dioxide provided a source of carbon for photosynthesis by diffusion through the permeable tube into the airspace as described above.

The culture vessel was stored in a temperature-controlled growth room, with the temperature controlled to around 23° C. The culture vessel was illuminated with a light source in the form of a white fluorescent tube of 36 W providing a light intensity of 100 μmol m⁻² s⁻¹ PAR. The light source provided a heat source to stir the liquid medium as described above.

FIG. 10 shows the apparatus 1000 used for the trial. The apparatus 1000 shows the culture vessel 1002 with Sphagnum 1006 arranged within the liquid medium 1004.

The Sphagnum was cultured in the culture vessel 1002 for 12 weeks.

Results

FIG. 11 shows the apparatus 1100 which corresponds to the apparatus 1000 of FIG. 10 after 12 weeks of culturing. The apparatus 1100 of FIG. 11 shows the culture vessel 1002 containing the liquid medium 1004. The Sphagnum 1106 has undergone significant growth compared to the Sphagnum 1006 of FIG. 11. After the 12 weeks of growth, around four times increase in growth of the Sphagnum was achieved.

1. An apparatus for use in culturing Sphagnum, comprising:
a culture vessel for Sphagnum;
a culture medium arranged in the culture vessel, wherein the culture vessel comprises an enclosed airspace above the culture medium; and
Sphagnum arranged in the culture medium;
wherein the apparatus is configured to supply carbon dioxide into the enclosed airspace.

2. The apparatus according to claim 1, wherein the apparatus is configured to supply a gas comprising at least 1% carbon dioxide by volume into the enclosed airspace.

3. The apparatus according to claim 1, wherein the apparatus is configured to supply a gas comprising at least 90% carbon dioxide by volume into the enclosed airspace.

4-6. (canceled)

7. The apparatus according to claim 1, further comprising a barrier permeable to carbon dioxide.

8. (canceled)

9. The apparatus according to claim 7, wherein the apparatus is configured to supply carbon dioxide through the barrier permeable to carbon dioxide and into the enclosed airspace.

10. The apparatus according to claim 7, wherein the barrier is arranged at least partially in contact with the enclosed airspace.

11. The apparatus according to claim 7, wherein the barrier is arranged at least partially within the culture vessel.

12. The apparatus according to claim 7, wherein the barrier comprises a tube permeable to carbon dioxide.

13. The apparatus according to claim 12, wherein the tube is arranged at least partially in the enclosed airspace.

14. The apparatus according to claim 12, further comprising an inlet pipe connected to a source of carbon dioxide, and wherein a first end of the tube is connected to the inlet pipe.

15. The apparatus according to claim 14, further comprising an outlet pipe connected to a second end of the tube.

16. The apparatus according to claim 1, wherein the culture medium does not comprise sugar.

17. The apparatus according to claim 1, wherein the culture medium comprises a liquid culture medium, and wherein the apparatus is configured to stir the liquid culture medium.

18. (canceled)

19. The apparatus according to claim 17, wherein the apparatus is configured to stir the culture medium from externally of the culture vessel.

20. The apparatus according to claim 17, further comprising a heat source configured to apply a temperature differential in the culture vessel to stir the liquid culture medium.

21. The apparatus according to claim 20, wherein the heat source comprises a light source.

22. The apparatus according to claim 1, further comprising a second culture vessel for Sphagnum and a second culture medium arranged in the second culture vessel, wherein the second culture vessel comprises a second enclosed airspace above the second culture medium, and further comprising Sphagnum arranged in the second culture medium; and
wherein the apparatus is configured to supply carbon dioxide into the second enclosed airspace of the second culture vessel.

23. The apparatus according to claim 22, wherein the culture medium comprises a liquid culture medium, wherein the apparatus is configured to stir the liquid culture medium, wherein the second culture medium comprises a second liquid culture medium, and wherein the apparatus is further configured to stir the second liquid culture medium of the second culture vessel.

24. The apparatus according to claim 23, further comprising a heat source configured to apply a temperature differential in the culture vessel and the second culture vessel to stir the liquid culture medium and the second liquid culture medium, wherein the heat source comprises a light source, and wherein the light source is arranged between the culture vessel and the second culture vessel.

25-26. (canceled)

27. A method of culturing Sphagnum, comprising:
providing a culture vessel for Sphagnum;
providing a culture medium arranged in the culture vessel, wherein the culture vessel comprises an enclosed airspace above the culture medium;
providing Sphagnum arranged in the culture medium; and
supplying carbon dioxide into the enclosed airspace.

28-31. (canceled)
